(19) United States
(12) Patent Application Publication
(10) Pub. No.: US 2022/0160301 A1
(43) Pub. Date: May 26, 2022

(54) TECHNIQUES FOR DETERMINING A HEALING STATUS OF AN ANATOMICAL STRUCTURE

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Joseph Ferrante, Bartlett, TN (US); Henry Faber, Memphis, TN (US); Darren J. Wilson, Hull (GB); Sied W. Janna, Memphis, TN (US)

(21) Appl. No.: 17/602,675

(22) PCT Filed: Apr. 8, 2020
(86) PCT No.: PCT/US2020/027252

§ 371 Date: Oct. 8, 2021

Related U.S. Application Data
(60) Provisional application No. 62/832,694, filed on Apr. 11, 2019.

(51) Int. Cl. A61B 5/00; A61B 17/72; A61B 5/11; G16H 40/63

(52) U.S. Cl. CPC A61B 5/686; A61B 17/72; A61B 5/0031; A61B 5/4842; A61B 2562/0261; A61B 5/112; G16H 40/63; A61B 2560/0214; A61B 5/4504

(57) ABSTRACT

A medical implant device such as, for example, an intramedullary (IM) nail including an improved sensing device for monitoring the progression of fracture healing in a patient is disclosed. In one embodiment, a medical implant device for attachment to a portion of a bone may include at least one energy-harvesting strain sensor operative to generate a charge responsive to a strain force on the medical implant device, and at least one electronic element configured to receive at least a portion of the charge, the at least one electronic element comprising a telemetry device operably coupled to the at least one energy-harvesting sensor, the telemetry device powered, at least in part, by the at least a portion of the charge to transmit sensor information associated with the charge to a receiver device. Other embodiments are described.

### CROSS-REFERENCE TO RELATED APPLICATION

This is a non-provisional of, and claims the benefit of the filing date of, pending U.S. provisional patent application No. 62/832,694, filed Apr. 11, 2019, entitled “Bone Fixation Device with Improved Sensing for Continuous Monitoring,” which application is incorporated by reference herein in its entirety.

### FIELD OF THE INVENTION

The present disclosure generally relates to medical implant devices, and, more particularly, to devices associated with sensors operative to acquire patient information relating to an anatomical structure, and processes for determining a healing status of the anatomical structure based on the patient information.

### BACKGROUND

Determining the progress of healing is an important part of the recovery process for patients that have undergone a serious medical condition, such as a surgical procedure, a broken bone, and/or the like. Conventional processes for examining the healing status of a patient typically involve direct investigation by a physician or other healthcare professional.

For example, monitoring the progress of fracture healing in a patient is usually assessed using a combination of patient testimony, clinical examination, and periodical X-rays. However, subjecting a patient to repeated X-rays can be a health concern. In addition, X-rays may not be reliable because interpretation is highly dependent on experience, leading to relatively poor inter-observer and intra-observer reliability, and partly because of the lack of an accepted definition of radiographic union. Moreover, the information gathered during an X-Ray examination is not strictly related to the mechanical properties of the healing fracture site. Patient testimony and clinical examination are inefficient, prone to error, and require clinical visits. In addition, these techniques are not able to directly examine the healing region, leading to inaccurate assessments.

Serious bone fractures typically require a bone fixation device (i.e., an implant) to support the patient's weight with an appropriate amount of stability to allow the surrounding fractured bone region to heal. After a fracture has been treated with a bone fixation device, the bone typically recovers its functionality over time and eventually absorbs most of the load in the bone/implant system. Knowledge of these properties is important to the patient and healthcare provider in terms of rehabilitation, potential secondary treatment, and determining the optimal time to return to normal activities.

Fracture healing can be monitored in real-time using an array of implantable strain gauges attached to the bone fixation device, with the strain on the bone fixation device continuously decreasing during a normal healing progress. As such, measuring the strain on the bone fixation device provides an objective clinical measure for a patient return to normal weight bearing and monitoring activity that could potentially place the bone fixation device or surgery at risk for biomechanical failure. For example, it has been determined that the risk of failure is 15 times higher in patients that did not follow recommended postoperative restrictions and/or when experimentally measured deformation was above the fatigue limit for the bone fixation device material.

Existing attempts to use strain gauges in bone fixation devices have required extensive changes to the design of the bone fixation device to accommodate the sensors and electronics that enable power and telemetry. For example, sensors generally need to be positioned within a machined recess in an outer surface of the bone fixation device to prevent the sensors from being subjected to excessive mechanical damage associated with abrasion forces, packaged in a biocompatible material such as epoxy resin or silicone rubber due to their potential toxic effects to mammalian cells, hermetically sealed within a welded cavity to prevent them from being damaged by excessive moisture, and/or positioned close to the fracture site (or sufficiently far away from the neutral axis) in order to maximize their sensitivity to changes in load strain during fracture healing.

In addition, the sensor and telemetry system have to be miniaturized to fit within the footprint of standard bone fixation devices such as, for example, an intramedullary (IM) nail, without modifications that could jeopardize the performance or reduce the effectiveness of the bone fixation device at its primary job, supporting the bone structure during healing. Conventional systems have attempted to provide long-term power systems, for example, in the form of enhanced and safer battery technology, unobtrusive electromagnetic induction via a miniaturized wearable reader device, or alternative approaches such as an implantable battery or energy harvesting from either applied biomechanical forces, ambient RF signals/backscatter, solar, body temperature or vibration/kinetic movement. However, the additional cost of these modifications has in conventional devices has not been low enough to make these devices commercially competitive and, therefore, a valuable tool of healthcare providers.

For these reasons among others, a need remains for further improvements in this technological field. The present disclosure addresses this need.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

In accordance with various aspects of the described embodiments is a medical implant device for attachment to a portion of a bone. The medical implant device may include at least one energy-harvesting strain sensor operative to generate a charge responsive to a strain force on the medical implant device, and at least one electronic element configured to receive at least a portion of the charge, the at least one electronic element may include a telemetry device operably coupled to the at least one energy-harvesting sensor, the telemetry device powered, at least in part, by the at least a portion of the charge to transmit sensor information associated with the charge to a receiver device.

In some embodiments of the medical implant device, the medical implant device may be formed as an intramedullary (IM) nail. In various embodiments of the medical implant device, the at least one energy-harvesting strain sensor may include a piezoelectric polyvinyl fluoride (PVDF) film sensor. In some embodiments of the medical implant device, the at least one electronic element may include at least one of a capacitor or a floating gate (FG) circuit. In various embodiments of the medical implant device, the sensor information may include at least one of: capacitor information associated with the capacitor indicating a charge stored on the capacitor, FG information associated with the FG circuit indicating at least one of a charge stored on the FG circuit, a number of channels storing a charge, or an injection rate, or telemetry charge information indicating a charge status of the telemetry device.

In exemplary embodiments of the medical implant device, the at least one energy-harvesting strain sensor may include a piezoelectric polyvinyl fluoride (PVDF) film sensor electrically coupled to a floating gate (FG) circuit, the charge to power the FG circuit. In some embodiments of the medical implant device, the medical implant device may include an intramedullary (IM) nail having a cannulation, the at least one energy-harvesting strain sensor may include a film arranged within the cannulation. In various embodiments of the medical implant device, the at least one energy-harvesting strain sensor may be formed as a cylinder to correspond to the cannulation, the cylinder may include a flexible polyimide circuit and electrode layers surrounding the at least one energy-harvesting strain sensor formed from a piezoelectric polyvinyl fluoride (PVDF) film.

In some embodiments of the medical implant device, the medical implant device may include a cap configured to store the at least one electronic element electrically coupled to the at least one energy-harvesting strain sensor. In some embodiments of the medical implant device, the medical implant device may include a pocket formed in an outer surface of the medical implant device, the at least one energy-harvesting strain sensor arranged within the pocket, and a lid to hermetically seal the pocket.

In accordance with various aspects of the described embodiments is a method of determining a healing status of a bone structure of a patient. The method may include determining a step count for the patient, determining sensor information associated with at least one energy-harvesting strain sensor of a medical implant device in contact with the bone structure, the at least one energy-harvesting strain sensor may be operative to: generate a charge responsive to a strain force on the medical implant device, and provide at least a portion of the charge to at least one electronic element, the at least one electronic element may include a telemetry device operably coupled to the at least one energy-harvesting sensor, the telemetry device may be powered, at least in part, by the at least a portion of the charge to transmit the sensor information associated with the charge to a receiver device, and determining the healing status based on the step count and the sensor information.

In some embodiments of the method, the at least one electronic element may include at least one of a capacitor or a floating gate (FG) circuit. In various embodiments of the method, the sensor information may include at least one of capacitor information associated with the capacitor indicating a charge stored on the capacitor, FG information associated with the FG circuit indicating at least one of a charge stored on the FG circuit, a number of channels storing a charge, or an injection rate, or telemetry charge information indicating a charge status of the telemetry device.

In some embodiments of the method, the method may include determining at least one value of expected sensor information for the step count based on healing information, and determining the healing status as a healing stage corresponding to the at least one expected value of sensor information. In some embodiments of the method, the at least one value of expected sensor information may include at least one of a capacitor charge, a telemetry device charge status, a number of charged FG channels, or an FG injection rate.

In accordance with various aspects of the described embodiments is an apparatus that may include a storage device and logic, at least a portion of the logic implemented in circuitry coupled to the storage device to implement a healing status analysis process. The logic may be operative to determine a step count for the patient, determine sensor information associated with at least one energy-harvesting strain sensor of a medical implant device in contact with the bone structure, the at least one energy-harvesting strain sensor operative to generate a charge responsive to a strain force on the medical implant device, and provide at least a portion of the charge to at least one electronic element, the at least one electronic element may include a telemetry device operably coupled to the at least one energy-harvesting sensor, the telemetry device powered, at least in part, by the at least a portion of the charge to transmit the sensor information associated with the charge to a receiver device, and determine the healing status based on the step count and the sensor information.

In some embodiments of the apparatus, the at least one electronic element comprising at least one of a capacitor or a floating gate (FG) circuit. In various embodiments of the apparatus, the sensor information may include at least one of capacitor information associated with the capacitor indicating a charge stored on the capacitor, FG information associated with the FG circuit indicating at least one of a charge stored on the FG circuit, a number of channels storing a charge, or an injection rate, or telemetry charge information indicating a charge status of the telemetry device.

In exemplary embodiments of the apparatus, the logic may operate to determine at least one value of expected sensor information for the step count based on healing information, and determine the healing status as a healing stage corresponding to the at least one expected value of sensor information. In some embodiments of the apparatus, the at least one value of expected sensor information may include at least one of a capacitor charge, a telemetry device charge status, a number of charged FG channels, or an FG injection rate.

### BRIEF DESCRIPTION OF THE DRAWINGS

FIG. 1 depicts an example of a first operating environment that may be representative of some embodiments of the present disclosure;

FIG. 2 depicts an example of a second operating environment that may be representative of some embodiments of the present disclosure;

FIGS. 3A and 3B depict example circuitry that may be representative of some embodiments of the present disclosure;

FIG. 4 depicts a graph of sensor information correlated with stages of fracture healing in accordance with aspects of the present disclosure;

FIG. 5 depicts an example of a third operating environment that may be representative of some embodiments of the present disclosure;

FIG. 6 depicts an example of a medical implant device having a plurality of mounting elements that may be representative of some embodiments of the present disclosure;

FIG. 7 depicts an example of a sensor device having cannulation mounting element that may be representative of some embodiments of the present disclosure;

FIGS. 8A and 8B depict examples of medical implant devices that may be representative of some embodiments of the present disclosure;

FIGS. 9A and 9B depict examples of a cap mounting element that may be representative of some embodiments of the present disclosure;

FIGS. 10A-10C depict examples of an external cavity mounting element that may be representative of some embodiments of the present disclosure;

FIG. 11 depicts a first example of a secondary power supply that may be representative of some embodiments of the present disclosure;

FIG. 12 depicts a second example of a secondary power supply that may be representative of some embodiments of the present disclosure;

FIG. 13 depicts an example of a terminal ring electrical connection for a sensor device that may be representative of some embodiments of the present disclosure;

FIG. 14 depicts an example of a electrical connection for connecting a sensor device to a device arranged within a cap mounting element that may be representative of some embodiments of the present disclosure;

FIGS. 15-17 depict schematic illustrations of examples of energy-harvesting polyvinyl fluoride (PVDF) film sensor devices that may be representative of some embodiments of the present disclosure;

FIGS. 18A-18C depict examples of PVDF film sensor devices that may be representative of some embodiments of the present disclosure;

FIG. 19 depicts an example of a cylindrical sensor that may be representative of some embodiments of the present disclosure;

FIG. 20 depicts a cross-sectional side view of a portion of a cannulated medical implant device that may be representative of some embodiments of the present disclosure;

FIG. 21 depicts a cross-sectional side view of a medical implant device that may be representative of some embodiments of the present disclosure;

FIG. 22 illustrates a logic flow in accordance with the present disclosure; and

FIG. 23 illustrates an embodiment of a computing architecture in accordance with the present disclosure.

The drawings are not necessarily to scale. The drawings are merely representations, not intended to portray specific parameters of the disclosure. The drawings are intended to depict example embodiments of the disclosure, and therefore should not be considered as limiting in scope. In the drawings, like numbering represents like elements.

Furthermore, certain elements in some of the figures may be omitted, or illustrated not-to-scale, for illustrative clarity. The cross-sectional views may be in the form of “slices”, or “near-sighted” cross-sectional views, omitting certain background lines otherwise visible in a “true” cross-sectional view, for illustrative clarity. Furthermore, for clarity, some reference numbers may be omitted in certain drawings.

### DETAILED DESCRIPTION

For the purposes of promoting an understanding of the principles of the present disclosure, reference will now be made to the embodiments illustrated in the figures and specific language will be used to describe the same. It will nevertheless be understood that no limitation of the scope of the disclosure is thereby intended. Any alterations and further modifications in the described embodiments, and any further applications of the principles of the present disclosure as described herein are contemplated as would normally occur to one skilled in the art to which the disclosure relates.

Various features, aspects, or the like of a healing status analysis system and method will now be described more fully hereinafter with reference to the accompanying drawings, in which one or more aspects of the healing status analysis system and/or method will be shown and described. It should be appreciated that the various features, aspects, or the like may be used independently of, or in combination, with each other. It will be appreciated that the healing status analysis system and method as disclosed herein may be embodied in many different forms and should not be construed as being limited to the embodiments set forth herein. Rather, these embodiments are provided so that this disclosure will convey certain illustrations of aspects of the healing status analysis system and method to those skilled in the art.

The present disclosure is directed to devices, systems, and methods for allowing efficient and accurate determination of a healing status of a portion of a human body. In some embodiments, a healing status analysis system may include or may be in communication with a sensor system. The sensor system may be configured to generate information that may be used by the healing status analysis system to determine a healing status of an effected portion of the human body, such as a fracture site. A medical implant device (for example, a bone-fixation device, such as an intramedullary (IM) nail) may be installed in or on a portion of a human body (for example, a femur). The sensor system may include a strain sensor coupled to the medical implant device. The strain sensor may be an energy-harvesting, self-powering sensor configured to generate a charge responsive to strain imparted on the portion of the human body. For example, the strain sensor may be a piezoelectric device, for example, a polyvinylidene fluoride (PVDF) or a copolymer of PVDF device. The charge may be stored in a capacitor, used to power a telemetry device, and/or used to power other devices or functions (such as a floating gate (FG) circuit). The sensor system may also include one or more patient sensors configured to measure physiological and/or activity aspects of a patient (for instance, a step count of the patient). In various embodiments, the charge generated by the strain sensor may be used alone or in combination with information from the patient sensors to determine a healing status of the portion of the human body. Accordingly, in some embodiments, a healing status analysis system may operate using a self-powered strain sensor to determine a progress of healing of a patient, for example, by harvesting energy from microscale strain variations in the medical implant device to allow for continuous and/or burst monitoring during the healing period.

In some embodiments, the energy-harvesting sensor may generate a charge in response to the stresses imposed on a bone fixation device, which in turn may power an implanted telemetry unit in order to transmit the charge generated by the strain sensor to an external, remote computing device, logic device, or other receiver unit. In various embodiments, the strain signal generated by the strain sensor, which corresponds to the amount of strain measured by the strain sensor, can be directly measured by discrete signal monitoring via the external computing device.

In one embodiment, the medical implant device may include a self-sensing bone fixation device or implant (used interchangeably without the intent to limit) such as, for example, an IM nail. The bone fixation device (e.g., IM nail) may include an implantable energy-harvesting piezoelectric sensor such as, for example, a PVDF or a copolymer of PVDF that are used in piezoelectric applications. In one embodiment, the bone fixation device may include a cannulation and/or external cavity and the PVDF sensor can be attached to an inside surface of the cannulation or external cavity. Although PVDF or a copolymer of PVDF are used in examples in the present disclosure, embodiments are not so limited, as other materials capable of operating according to some embodiments are contemplated in the present disclosure.

In general, a fracture site is different for each individual trauma case. In addition, a fracture site is also dependent on a patient's size and other physical characteristics. Accordingly, in some embodiments, a bone fixation device may include a cannulation and a single sensor that extends across an entire length or substantially the entire length of the cannulation. Thus arranged, the need to incorporate multiple sensors deployed along the length of the bone fixation device, as required in conventional devices, to ensure that at least one sensor is always located at or close to the fracture site for maximum sensitivity for detecting load changes on the bone fixation device during fracture healing is mitigated.

In one embodiment, the strains measured on the bone fixation device could be determined from the number of steps required to power a wireless electromagnetic signal to an external receiver unit through the amount of charge stored on the generator storage circuit. For example, walking or exercise will induce stresses upon the fractured limb, which can then be picked up by the strain sensor unit affixed to a medical implant device configured according to some embodiments.

In some embodiments, fracture healing can be interpreted from the outputs of an FG device, such as miniaturized FG circuit board. In one embodiment, the FG circuit board may be a 1.5 mm×1.5 mm (or similar) in size using complementary metal-oxide-semiconductor (CMOS) technology. Time-evolution curves and histograms produced from the floating gate circuitry may be used to differentiate between different conditions of healing. For example, in one embodiment, a histogram may typically represent the number of channels that are injecting into the floating gate memory cell at any given time. As bone healing progresses, the number of injecting channels and/or the injection rate of the FG circuit may decrease. The FG may also act as a non-volatile memory for storage from which data from the bone fixation device can be retrieved offline by an external reader for future analysis, thereby avoiding any “black-outs.” A PVDF sensor can also be programmed to record statistics of different levels of strain or different rates of strain by modifying the interface circuit. Accordingly, a healing status analysis system described in this disclosure may be able to record instantaneous changes in strain levels from a medical implant device (e.g., an IM nail), eliminating the need for external receiver for continuous data logging.

Contrary to known prior art sensors for measuring bone healing that typically rely on embedded foil strain gauges, which, among other issues, require relatively high power requirements and use between 500 mW of battery power to 5 watts of induced external power, the power requirements of sensor systems configured according to some embodiments may use significantly less power (for instance, less than 1 mW, especially with the support of an FG sensor circuit).

In one embodiment, a strain sensor may be a self-powered, self-sensing biocompatible PVDF or a copolymer of PVDF that exhibits piezoelectric energy harvesting properties and can provide continuous power when attached to an orthopedic implant such as an IM nail. Contrary to existing sensors, that suffer from limited life and complications due to biocompatibility, a piezoelectric based self-powering sensor according to the present disclosure may be biocompatible (USP Plastic Class VI) and abrasion resistant. As such, a strain sensor configured according to some embodiments may not require a complex hermetic assembly for adequate insulation and protection from body fluids. In one embodiment, a strain sensor configured according to some embodiments can also be sterilized by autoclave (e.g., 121° C. at 1 bar), Gamma irradiation, and/or Ethylene oxide (ETD). Sensor materials according to some embodiments may be formable to the bone fixation device at a low cost compared to existing sensors. Contrary to conventional piezo-electric ceramics such as PZT, PVDF sensors may eliminate the use of lead and are not susceptible to brittle failure, thus substantially reducing, if not completely eliminating, the risk of an adverse toxicological response.

In one example embodiment, a strain sensor (e.g., a PVDF sensor) may be arranged and configured to extend across an entire length of the medical implant device, such as an IM nail, negating the need for multiple sensors deployed along the IM nail to ensure that at least one sensor is always located at the fracture site for maximum sensitivity to detecting strain changes in the medical implant device during bone healing. In addition, and/or alternatively, the strain sensor can be located within the cannulation or “dead space” of the IM nail reducing the risk of mechanical damage from the neighboring bone during implantation.

In addition, and/or alternatively, the primary sensor may not require significant modification to the existing bone fixation device, thereby simplifying the manufacturing process. Moreover, as described in more detail below, ancillary electronics can be located in a cap or other mounting element removably coupled to the IM nail. In some embodiments, the cap can be specifically designed to minimize attenuation of wireless (e.g., RF) signals.

PVDF sensors without FG circuitry may require more power (for example, 100 times more power) than what is generally available under normal physiological conditions. In addition to ultralow power levels, the occurrence of loading cycles for FG devices may be infrequent, necessitating integration of self-powered computation and sensing with non-volatile storage. Piezoelectric transducers may generate large voltage swings (5 V), but at nanoamperes current level. This property is ideal for triggering avalanche injection in FG transistors, which can be used to achieve sensing, computation and storage at power levels less than 1 μW. FG transistor circuitry configured according to some embodiments may eliminate the need for voltage regulation, energy storage, analogue-to-digital converters, microcontroller, units, and random access memories further simplifying the assembly process. In addition, FG injector/transistor circuits may allow a bone fixation device configured according to some embodiments to be continuously or semi-continuously monitored for a long duration (e.g., 3 months or more) using <1 μW of power without the need for a secondary energy source, such as a wireless charging unit (inductive or magnetic field) or a battery cell.

In one embodiment, a PVDF strain sensor can be formed as a thin film, which may provide the sensor with a higher piezoelectricity and sensitivity. In addition, a strain PVDF sensor may potentially fill the entire or substantially the entire inside surface of a bone fixation device. In addition, a removable cap may be used to house a secondary power source and/or other devices to facilitate continuous monitoring for other sensors (e.g., thermistor, accelerometer, etc.) and/or a low energy telemetry (e.g., Bluetooth chip) for transmitting data wirelessly to an external receiver unit.

In some embodiments, the strain sensor and patient sensors may be used during a bone healing process to determine a healing status through either (a) correlating the amount of energy stored on a capacitor and the number of steps used to power a simple RF link, or (b) the number of injecting channels as well as the injection rate of a floating gate sensor circuit.

Healing status analysis processes according to some embodiments may provide multiple technological advantages and technical features over conventional systems, including improvements to computing technology. One non-limiting example of a technological advantage may include efficient and accurate determination of a healing status of a patient using a combination of patient information (for instance, step count) and sensor information (for example, capacitor information, FG information, and/or telemetry charge status information). A problem in computing technology for existing systems involves the ability to present healing information on a computing display using low-power medical implant devices that do not require secondary power sources (e.g., batteries, induction power, etc.). Some embodiments may provide an improvement in computing technology by facilitating presentation of a healing status on a remote computing device using completely self-powered, energy harvesting medical implant devices through the novel combined use of patient information and sensor information. Other technological advantages are provided by various embodiments and are described in the present disclosure.

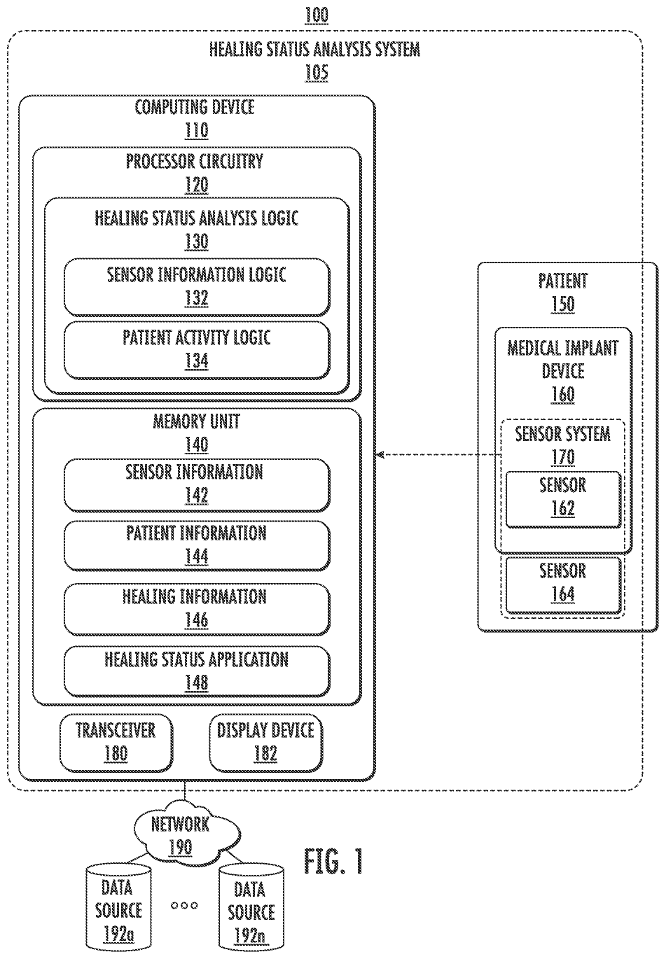

FIG. 1 illustrates an example of an operating environment 100 that may be representative of some embodiments. As shown in FIG. 1, operating environment 100 may include a healing status analysis system 105. In various embodiments, healing status analysis 105 may include a computing device 110 communicatively coupled to network 190 via a transceiver 180. Computing device 110 may be or may include one or more logic devices, including, without limitation, a server computer, a client computing device, a personal computer (PC), a workstation, a laptop, a notebook computer, a smart phone, a tablet computing device, and/or the like. Embodiments are not limited in this context.

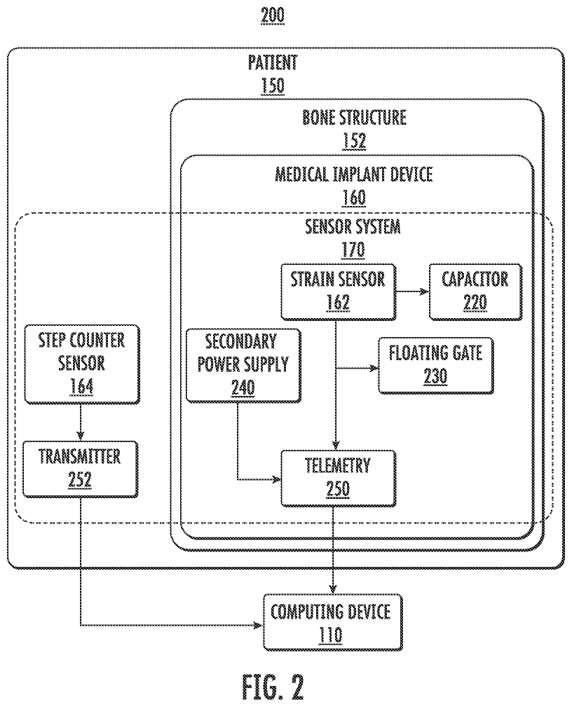

As shown in FIG. 1, healing status analysis system 105 may include or may be communicatively coupled to a sensor system 170. FIG. 2 illustrates an example of an operating environment 200 that may be representative of some embodiments. More specifically, FIG. 2 depicts a detailed illustration of sensor system 170 within operating environment 200.

With reference to FIGS. 1 and 2, in some embodiments, healing status analysis system 105 may include a medical implant device 160 configured to be implanted in or on a portion 152 of a patient 150. A non-limiting example of a portion of the human body 152 may include a bone structure, such as a femur, a knee joint, a hip joint, and a tibia. In some embodiments, medical implant device 160 may include a bone fixation device, such as an intramedullary (“IM”) nail, hip implant, knee implant, tibial tray, or other orthopedic implant. Although bone anatomy and an IM nail are used in examples in the present disclosure, embodiments are not so limited, as any portion of the human anatomy and/or any type of medical implant device capable of being used according to some embodiments are contemplated herein.

Medical implant device 160 may be associated with at least a portion of a sensor system 170 having a strain sensor (or strain gauge) 162 configured to generate strain information that may be correlated with a healing status of the portion of the human body 152. For example, an IM nail implanted in a bone may be coupled to a strain sensor configured to generate a charge (voltage, strain information, or strain signal) responsive to a strain or load applied to the IM by the bone, for example, as a result of the patient walking or otherwise putting weight on the effected bone. Strain sensor 162 may generate strain information 142 or a strain signal, such as a voltage or other signal, indicating the amount and/or frequency of strain experienced by medical implant device 160. With respect to strain sensor 162, the terms strain information, strain signal, charge, and voltage may be used interchangeably herein without the intent to limit the disclosure, unless specified otherwise. In some embodiments, strain information 142 may be used by healing status analysis system 105, at least in part, to determine a healing status or healing information 146 for an effected portion of the human body.

In some embodiments, strain sensor 162 may be or may include an energy-harvesting and/or self-powered strain sensor. For example, in one embodiment, strain sensor 162 may be an energy-harvesting piezoelectric sensor. In various embodiments, strain sensor 162 may be formed of polyvinylidene fluoride (PVDF), a PVDF copolymer, variations thereof, and/or combinations thereof. In some embodiments, strain sensor 162 may include or may be operably coupled to a capacitor 220. In various embodiments, at least a portion of the charge generated by strain sensor 162 may be stored in capacitor 220. In various embodiments, the amount of charge stored in capacitor 220 may be included as strain information 142, for example, as capacitor information or capacitor charge. In some embodiments, the capacitor information may be used by healing status analysis system 105, at least in part, to determine healing information 146 for an effected portion of the human body.

In exemplary embodiments, sensor system 170 may include a FG device 230. In various embodiments, FG device 230 may be coupled to strain sensor 162 to receive at least a portion of the charge generated by strain sensor 162. If the charge of strain sensor 162 transmitted to FG device 230 is over a FG barrier threshold, the charge may cause a FG charge to be generated in the FG device (i.e., by exciting high-energy or hot electrons in one or more channels of the FG device to become trapped in an FG gate). In some embodiments, the FG barrier threshold may be about 1 V, about 2 V, about 3 V, about 5 V, and any value or range between any two of these values (including endpoints). In various embodiments, FG information may be included as strain information 142, for example, as a number of injecting channels of the FG device, an injection rate, and/or the like. In some embodiments, the FG information may be used by healing status analysis system 105, at least in part, to determine healing information 146 of an effected portion of the human body.

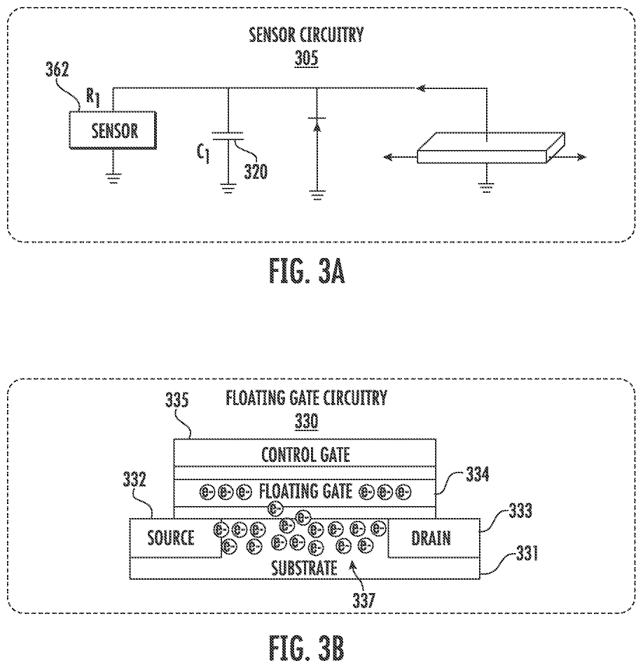

FIGS. 3A and 3B depict example circuitry that may be representative of some embodiments of the present disclosure. FIG. 3A depicts sensor circuitry 305 that may be representative of sensor 162. For example, sensor circuitry 305 may include a strain sensor 362 operably coupled to capacitor 320. In some embodiments, circuitry 305 may be a storage capacitor circuit, for example, a piezoelectric generator charge circuit. In various embodiments, a charge generated on strain sensor 362 may be collected during each load cycle on capacitor 320. In various embodiments, circuitry 305 may have a power requirement that is less than about 1 mW.

Referring to FIG. 3A, the relatively high and repetitive nature of loads applied during walking makes orthopedic an attractive area for energy harvesting. Under normal physiological walking, biomechanical loads may produce approximately 1200 microstrains (le) on a surface of an IM nail, which may correspond to approximately 14.5 V (open circuit) per step for a (5 cm×4 cm×0.0028 cm) (0.056 cm²) PVDF film. Using sensor circuit 305, this may correspond to approximately 0.20 per step or an average power of 0.16 μW. In some embodiments, the charge of sensor circuit 305 may be obtained continuously. In other embodiments, the charge of sensor circuitry 305 may be obtained during bursts, such as about every 3 hours. In various embodiments, the charge may be obtained during bursts about every 1 minute, about every 5 minutes, about every 10 minutes, about every 15 minutes, about every 30 minutes, about every 1 hour, about every 2 hours, about every 3 hours, about every 4 hours, about every 5 hours, about every 10 hours, and/or any value or range between any two of these values (including endpoints). For an average sized IM nail (e.g., 32 cm long×10 mm outer diameter×5.4 mm internal diameter), assuming a total surface area of (160.8 cm²) and an internal surface area of about 80 cm², the amount of power generated from a PVDF sensor 362 could be about 200-300 μW, which is within a typical power range for supplying energy to generic ultra-low power integrated circuits for biomedical applications.

Referring to FIG. 3B, therein is depicted FG circuitry 330 according to some embodiments. FG circuitry 330 may be used according to various embodiments to implement FG device 230. As shown in FIG. 3B, FG circuitry 330 may include a substrate 331, a source 332, a drain 333, a floating gate 334, and a control gate 335. When a charge is applied to FG circuitry 330, high-energy or hot electrons 337 are generated. If the energy of hot electrons 337 is over an FG barrier threshold, hot electrons 337 may enter (i.e., be injected into) floating gate 334. Because floating gate 334 is electrically isolated, hot electrons 337 that are injected into floating gate 334 may be trapped within floating gate 334.

For example, in one embodiment, a Floating-Gate MOS transistor (FGMOS), can be heterogeneously integrated into microsystem sensor platforms, such as sensor 162 and/or sensor circuitry 330, and can be used for low voltage/low power applications owing to its unique feature of programmability of threshold voltage, which can be lowered from its conventional value. A piezoelectric FG circuit has the advantage of being self-powered, low power consumption (e.g., less than 1 uW), requiring no maintenance, exhibiting a small form factor, for example, using semi-conductor CMOS technology, and low cost.

In one embodiment, FG device 230 may be a poly-silicon gate surrounded by an insulator oxide to ensure that it is electrically isolated by high-quality insulating oxide. Injected electrons (for instance, hot electrons 337) may remain trapped for a long period of time. As the piezoelectric element (for example, of sensor 162) is periodically excited, more electrons are injected onto the floating gate and the total amount of charge on the floating gate indicates the duration and extent of the mechanical strain exerted on the bone fixation device (for example, medical implant device 162). In some embodiments, FG device 230 may include a plurality of channels, for example, configured as a channel array. In various embodiments, channels in the channel array may have different FG barrier thresholds. FG circuitry 330 may eliminate the need for voltage regulation, energy storage, analogue-to-digital converters, microcontroller units, and random access memories.

Referring to FIGS. 1 and 2, in some embodiments, strain sensor system 170 may include a telemetry device 250, transmitter, transceiver, or other or element operative to transmit sensor information to a remote device, such as computing device 110. Telemetry device 250 may operate according to various wireless transmission protocols, including, without limitation, radio frequency (RF), Bluetooth™, Zigbee™, near field communication (NFC), Medical Implantable Communications Service (MICS), and/or the like. In various embodiments, the charge generated by strain sensor 162 may be used to at least partially to power telemetry system 250. In some embodiments, the charge status of telemetry system 250 (i.e., whether telemetry system 250 is or has been sufficiently charged to operate) may be included as healing information 146. In some embodiments, the charge status of telemetry system 250 may be used by healing status analysis system 105, at least in part, to determine healing information of an effected portion of the human body.

Accordingly, in some embodiments strain sensor 162 may be configured to generate a charge in response to strain imparted on medical implant device 160. At least a portion of the charge may be transmitted to one or more electronic elements or destination devices, such as capacitor 220, FG device 230, and/or telemetry device 250. The charge may be stored in capacitor 220 to generate capacitor information, transmitted to FG device 230 to generate FG information, used to power telemetry device 250, and/or transmitted to a remote device, such as computing device 110, as strain information 146 (e.g., for direct, discrete monitoring).

In various embodiments, patient 150 may be associated with one or more patient sensors 164 arranged outside of and independent from medical implant device 160. Patient sensors 164 may be configured to generate patient information 144, which may include, without limitation, physiological and/or activity-related information of patient 150. Non-limiting examples of patient sensors 164 may include Internet-of-Things (IOT) sensors, an accelerometer, a gyroscope, an altimeter, a global positioning system (GPS) sensor, a heart rate sensor, a pulse oximeter, temperature sensor, and/or the like. In various embodiments, patient sensors 164 may transmit data via transmitter 252 to computing device 110. In some embodiments, patient sensors 164 may operate to generate patient information 144 in the form of a step count of the patient. In some embodiments, patient information 144, such as a step count, may be determined using an application, for instance, executing on patient sensor 164 (for instance, an IoT or “fitness tracker” device) and/or a computing device (for instance, via a smartphone application in communication with patient sensor 164). In various embodiments, the step count required to be measured to perform a healing status analysis process (for instance, to have sufficient data) may be a large number (for example, greater than 500), such that an electronic sensor and associated software are necessary to carry out processes according to some embodiments.

As described in more detail below, a healing status analysis process may use patient information 144 in combination with sensor information 142 to determine healing information 146, for instance, a healing status or progress of patient 150. For example, a healing status analysis process may determine the healing stage of a fractured bone of patient 150 based on a step count of patient 150 and strain information 142, such as energy stored on capacitor 220, a number of steps required to power a portion of strain sensor 162, such as the telemetry device 230, and/or FG information (e.g., a charge stored at FG device 230, a number of injecting channels, and/or an injection rate).

In some embodiments, a healing status may include information indicating that there is abnormal, aberrant, unexpected, or otherwise an issue with the healing progress. For example, healing status application 148, for example, via healing status analysis logic 130, may determine or be instructed of a healing timeline (e.g., the patient should be at stage X by week Y) or other expectation of healing progress. Healing status application 148 may determine that the patient is experiencing abnormal healing, for example, because they are not at a particular stage at an expected time or because of another detected abnormality (e.g., elevated temperature, abnormal gait, and/or the like). In some embodiments, healing status application 148 may generate an abnormal healing status message, alert, or other signal indicating an abnormal healing condition. In various embodiments, healing status application 148 may determine a treatment recommendation based on sensor information 142, patient information 144, healing information 146, and/or the like for the abnormal healing status. For example, healing status application 148 may access a database of health information to determine a treatment recommendation based on available information. For instance, patient information 144 and/or sensor information 142 indicating a high temperature may indicate an infection and healing status application 148 may generate a treatment recommendation for patient 150 to be checked for an infection. In another instance, patient information 144 and/or sensor information 142 may indicate an abnormal gait for the expected stage of healing and generate a corresponding treatment recommendation.

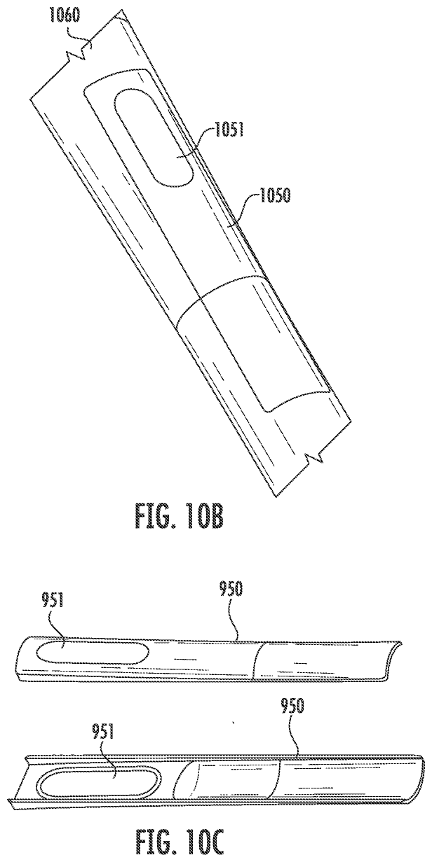

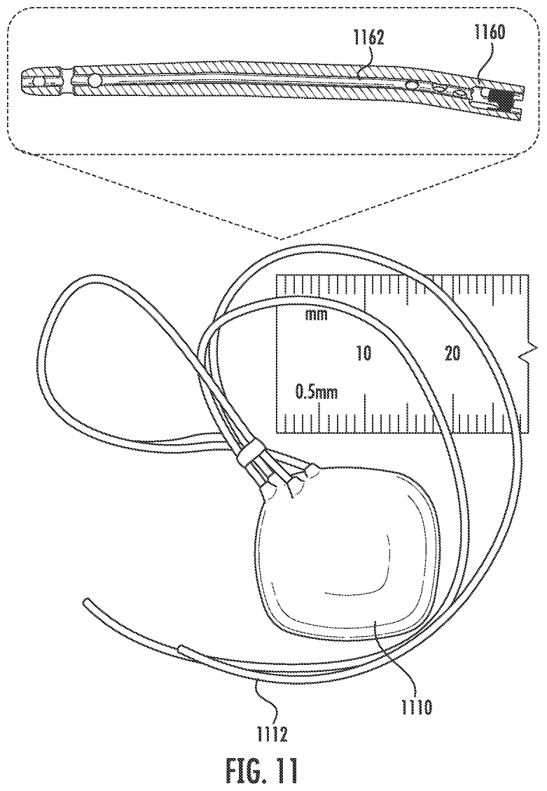

In some embodiments, a secondary power supply 240 may be associated with medical implant device 160 (see, for example, FIGS. 11 and 12). Secondary power supply 240 may include a battery, inductive charging device, magnetic field charging device, RF charging device, and/or the like configured to provide an alternative power supply for elements associated with strain sensor 162, such as to power telemetry 250. For example, due to the low mechanical-to-electrical coupling coefficients and non-uniform distribution of the strain along the medical implant device 160, sensor device 162, may generate a voltage (for instance, less than 1 V) that is less the amount required to power telemetry 250, necessitating the need for a secondary energy source such as, for example, a battery. Although secondary power supply 240 is included in some embodiments, strain sensor 162, telemetry device 250, and/or the like may be operate using the energy generated by strain sensor without requiring a secondary power supply. Embodiments are not limited in this context.

Computing device 110 may be configured to manage, among other things, operational aspects of a healing status analysis process according to some embodiments. Although only one computing device 110 is depicted in FIG. 1, embodiments are not so limited. In various embodiments, the functions, operations, configurations, data storage functions, applications, logic, and/or the like described with respect to computing device 110 may be performed by and/or stored in one or more other computing devices (not shown), for example, coupled to computing device 110 via network 170. A single computing device 110 is depicted for illustrative purposes only to simplify FIGS. 1 and 2. Embodiments are not limited in this context.

Computing device 110 may include a processor circuitry 120 that may include and/or may access various logics for performing processes according to some embodiments. For instance, processor circuitry 120 may include and/or may access a healing status analysis logic 130, a strain information logic 132, and/or a patient activity logic 134. Processing circuitry 120, healing status analysis logic 130, strain information logic 132, and/or patient activity logic 134, and/or portions thereof may be implemented in hardware, software, or a combination thereof. As used in this application, the terms “logic,” “component,” “layer,” “system,” “circuitry,” “decoder,” “encoder,” “control loop,” and/or “module” are intended to refer to a computer-related entity, either hardware, a combination of hardware and software, software, or software in execution, examples of which are provided by the exemplary computing architecture 2300. For example, a logic, circuitry, or a module may be and/or may include, but are not limited to, a process running on a processor, a processor, a hard disk drive, multiple storage drives (of optical and/or magnetic storage medium), an object, an executable, a thread of execution, a program, a computer, hardware circuitry, integrated circuits, application specific integrated circuits (ASIC), programmable logic devices (PLD), digital signal processors (DSP), field programmable gate array (FPGA), a system-on-a-chip (SoC), memory units, logic gates, registers, semiconductor device, chips, microchips, chip sets, software components, programs, applications, firmware, software modules, computer code, a control loop, a computational model or application, an AI model or application, an ML model or application, a proportional-integral-derivative (PID) controller, FG circuitry, variations thereof, combinations of any of the foregoing, and/or the like.

Although healing status analysis logic 132 is depicted in FIG. 1 as being within processor circuitry 120, embodiments are not so limited. For example, healing status analysis logic 130, strain information logic 132, patient activity logic 134, and/or any component thereof may be located within an accelerator, a processor core, an interface, an individual processor die, implemented entirely as a software application (for instance, a healing status application 148) and/or the like.

Memory unit 130 may include various types of computer-readable storage media and/or systems in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In addition, memory unit 130 may include various types of computer-readable storage media in the form of one or more lower speed memory units, including an internal (or external) hard disk drive (HDD), a magnetic floppy disk drive (FDD), and an optical disk drive to read from or write to a removable optical disk (e.g., a CD-ROM or DVD), a solid state drive (SSD), and/or the like.

Memory unit 130 may store various types of information and/or applications for a healing status analysis process according to some embodiments. For example, memory unit 130 may store strain information 142, patient information 144, healing information 146, and/or a healing status application 148. In some embodiments, some or all of strain information 142, patient information 144, healing information 146, and/or a healing status application 148 may be stored in one or more data stores 192a-n accessible to computing device 110 via network 190.

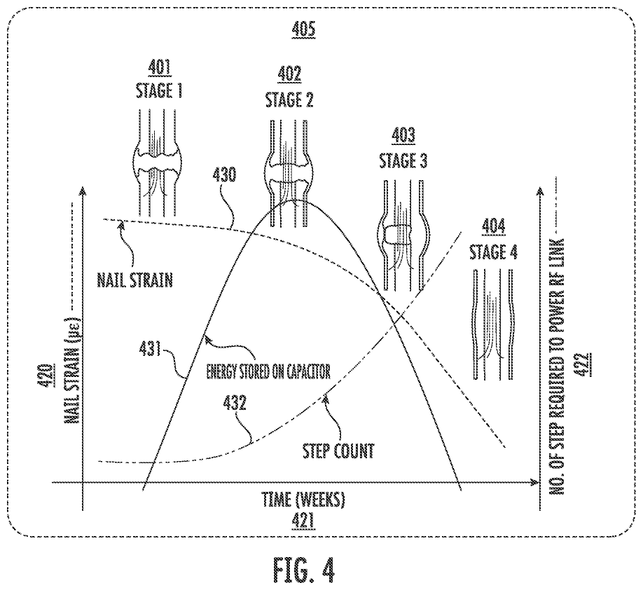

In some embodiments, healing status analysis logic 130, for example, via strain information logic 132, patient activity logic 134, and/or healing status application 148, may operate to analyze strain information 142 and/or patient information 144 to generate healing information 146. In various embodiments, healing information 146 may include a healing status of a portion of a human body, such as a stage of fracture healing (see, for example, FIG. 4).

In various embodiments, strain information 142 may include data, signals, and/or other information associated with strain sensor 162 and/or operation thereof. In some embodiments, strain information 142 may include a charge generated by strain sensor 162 (strain charge information), for example, responsive to a strain imparted on medical implant device 160. For example, the charge generated by strain sensor 162 (and/or information associated therewith) may be provided to healing status analysis logic 130 which may convert this strain charge information into healing information 146 (for instance, an estimate of a healing stage of bone structure 152).

In various embodiments, strain information 142 may include information associated with a charge stored in capacitor 220. For example, as strain sensor 162 generates a charge responsive to strain, at least a portion of the charge may be stored on capacitor 220. Capacitor 220 may lose some of this charge over time or as a result of use of the charge by the circuit and/or other devices. As healing progresses in bone structure 152, strain on medical implant device 160 decreases. Accordingly, sensor 162 generates less charge responsive to patient 150 activity and, therefore, the amount of charge stored on capacitor 220 may decrease. The amount of charge on capacitor 220 may be determined as capacitor information of sensor information 142. In some embodiments, capacitor information may be used, at least in part, to determine healing information 146.

In exemplary embodiments, strain information 142 may include FG information associated with FG device 230. Non-limiting examples of FG information may include a charge stored in FG device 230, channel-level charge information (for instance, a number of channels of a channel array storing a charge and/or which channels have a stored charge and the amount of the charge stored in each channel), an injection rate, and/or other information that may be obtained from FG device 230. The FG information may change over time as bone structure 152 heals. In some embodiments, the FG information may be used, at least in part, to determine healing information 146.

In some embodiments, strain information 142 may include charge status information of telemetry device 250. For example, healing status application 148, for example, via healing status analysis logic 130, may operate to determine whether telemetry device 250 is sufficiently charged (i.e., able to provide a signal to computing device 110). For instance, charge status information may include a “charged” status when telemetry device 250 has been sufficiently charged by sensor 162 (i.e., and can provide a signal to computing device) and a “non-charged” status otherwise (or below a threshold charge level). As described in more detail below, the charge status of telemetry device 250 may be used in combination with patient information 144 (for instance, step count) to determine a number of steps by patient 150 required to charge telemetry device 150. As healing progresses in bone structure 152, strain on medical implant device 160 decreases. Accordingly, sensor 162 generates less charge responsive to patient 150 activity and, therefore, the number of steps required to power telemetry device 250 increases.

In various embodiments, patient information 144 may include data, signals, or other information associated with physiological characteristics and/or activity of patient 150. Non-limiting examples of patient information 144 may include height, weight, age, gender, condition (i.e., bone fracture, healing stage, and/or the like), temperature, blood pressure, step count, calorie burn count, and/or the like. In some embodiments, patient information 144 may include the same or similar information for a population of individuals. For example, step count for patient 150 may be compared with step count for a similar population of patients, for example, to determine an expected number of steps for patient 150 to charge telemetry device 250 during various stages of healing. In some embodiments, patient information 144 may include models, such as machine learning (ML), neural network (NN), or other artificial intelligence (AI) models to model patient information 144 in combination with sensor information 142 to determine healing information 146.

In some embodiments, healing status application 148 may be or may include an application being executed on computing device 110 (including a mobile application or “app” executing on a mobile device form factor). Healing status application 148 may include or may be an application interface for healing status analysis logic 130 and/or components thereof. Healing status application 148 may receive sensor information 142 and/or patient information and may determine healing information 146. In some embodiments, healing status application 148 may present healing information 146 on display device 182.

In some embodiments, healing information 146 may include a diagnosis, estimate, prediction, or other information associated with the healing status of bone structure 152 (see, for example, FIG. 4). For example, a fracture progresses through certain phases during the healing process. Healing information 146 may include a diagnosis or estimate of which stage of healing patient 150 is in based on sensor information 142 and/or patient information 144.

FIG. 4 depicts a graph of sensor information correlating with stages of fracture healing in accordance with aspects of the present disclosure. The strains measured on a bone fixation device may be determined from the number of steps required to power a wireless electromagnetic signal to an external receiver unit through the amount of charge stored on the generator storage circuit. For example, walking or exercise will induce stresses upon the fractured limb, which can then be picked up by the sensor unit affixed to the bone fixation device

In general, FIG. 4 depicts graph 405 correlating four stages 401-404 of fracture healing with the amount of energy stored on a capacitor and the number of steps used to power a telemetry device, such as an RF link. The number of steps taken by the patient during rehabilitation, which are required to power the RF link, may directly indicate the mechanical force carried by the bone fixation device. As normal healing progresses, the bone carries more mechanical load and thus the number of steps to power the RF link increases.

In the early stage of fracture repair, referred to as stage 1 401 in FIG. 4, the patient would have undergone surgery and received an IM nail equipped with the primary sensor (e.g., strain sensor 162) that can detect changes in implant strain during loading, which may be interpreted through changes in charge stored on a capacitor circuit (e.g., capacitor 220 and/or circuit 305). Given that the patient is not ambulating during the immediate recovery period and is thus not subjecting peak forces on the injured limb, the amount of energy generated from the piezoelectric sensor may be marginal (for instance, insufficient to establish a charge on a capacitor and/or power a telemetry device) and may require a second energy source such as, for example, a battery or secondary energy harvesting and storage system to power the circuits.

As the patient starts to ambulate during the early stages of tissue repair, the primary sensor (strain sensor 162) on the bone fixation device (medical implant device 160) detects a high level of strain, which is registered from the forces exerted through the bone fixation device during rehabilitation. At this stage, the mechanical-to-electrical energy conversion is capable of powering a wireless electromagnetic signal (for instance, transmitted by telemetry device 250), which allows data to be telemetered to a remote device such as, for example, a mobile device. As the patient increases their mobility during rehabilitation, referred to as stage 2 in FIG. 4, the energy stored on a capacitor circuit increases. As the bone starts to heal, the amount of strain registered on the bone fixation device by the piezoelectric sensor decreases, referred to as stage 3 in FIG. 4, but the number of steps required to power the RF circuit increases. When the bone fully unites, referred to as stage 4 in FIG. 4, the strain registered on the bone fixation device could be less, for example, below approximately 50% of the amount measured after surgery. The amount of energy generated from the sensor and stored in the capacitor at this stage of rehabilitation would be lower (including a complete lack of charge stored in the capacitor) due to the number of steps required to power the circuitry, thus a secondary energy source could be used to provide additional charge to power the internal circuits continuously based on the sampling rate of the sensor.

In one embodiment, a bone fixation device may include a sensor, for example, a thin-filmed PVDF, which is an electro-active fluoro-polymer, due to its capability to elicit the piezoelectric effect. The piezoelectric effect allows the thin-filmed PVDF sensor to produce an electric charge when subjected to mechanical stress with higher piezoelectricity and sensitivity observed with thinner films (1 μm or less). Consequently, load changes in the bone fixation device can be correlated with fracture healing through the number of steps required to power an RF link to an external receiver as a corresponding strain measurement signal.

Referring to FIG. 4, graph 405 depicts stages 401-404 of fracture healing in combination with nail strain 420 and charge steps (number of steps to power a telemetry system, such as an RF link) versus time 421. As indicated by plot 430, the amount of nail strain decreases over time, while the charge steps 432 increases. Capacitor information 431 indicates that the energy stored on a capacitor (for instance, capacitor 220) increases early in the healing process (for instance, during stage 1 401 and early stage 2 402), peaks (for example, during stage 2 402), then decreases through the later stages (for example, stage 3 403 and/or stage 4 404, eventually reducing to zero.

In some embodiments, patient information 144 may include a step count of patient. The step count may be over a specific duration (a step count duration). For example, a patient sensor capable of determining step count information may provide step count information to computing device 110. In various embodiments, the patient information 144 may include one or more step conversion factors, for example, to convert, estimate, or otherwise determine a number of steps to correlate to an expected amount of a corresponding item of sensor information. For example, a telemetry charge conversion factor may convert an expected number of steps to charge a telemetry device, such as an RF link (such as converting μJ/step to a voltage required to power a telemetry device). For instance, the telemetry charge conversion factor may determine that for fracture healing stage 2, 500 steps are required to charge an RF link (i.e., if the patient takes 500 steps, the RF link should have a charged status), for stage 3, 1000 steps are required to charge the RF link, and so on. In another example, a capacitor conversion factor may estimate a capacitor charge for a certain number or range of steps. For instance, if a patient takes 500 steps during stage 2, the capacitor charge should be 1 coulomb (or volt, Farad, or other capacitance unit), if a patient takes 1000 steps during stage 2, the capacitor charge should be 1.5 coulombs, if a patient takes 500 steps during stage 4, the capacitor charge should be 0.25 coulombs, and so on. In another example, an FG conversion factor may be configured to correlate steps taken to FG charge characteristics over the various stages of healing, such as number of channels, charge value, injection rate, and/or the like.

In some embodiments, the step count conversion factors may be based on patient-specific factors, such as gender, height, weight, fracture characteristics, and/or the like. In various embodiments, patient information 144 may include population-based information that includes step conversion factors for various patients. In this manner, the step conversion factors for healing status application 148 for a specific patient may be set to correspond with step conversion factors for a patient population correlating with the patient. For instance, for a male patient, aged 40, with a height of 6 feet and a weight of 250 pounds, the step conversion factors for healing status application 148 may be set to values determined or estimated based on a corresponding patient population.

In some embodiments, use of sensor information 142 may be weighted for the different stages of healing. For example, telemetry charge information may be more accurate during the early stages of healing (for example, stages 1 and 2), but may be less reliable during later healing stages. In another example, capacitor charge information may be downgraded by giving capacitor charge information a low weight (including zero) during stage 1. Accordingly, in some embodiments, sensor information may be adjusted by a sensor information weight factor for each stage. Embodiments are not limited in this context. In some embodiments, ML, NN, and/or other AI models may be used to model or generate step conversion factors and/or determine sensor information weight factors.

Although stages 1-4 of a fracture healing process are used as an example in the present disclosure, embodiments are not so limited. For example, step count information may be correlated to various different healing, staging, or other progressions. For instance, as long as step conversion factors are determined for each specific stage, healing status application 148 may determine a healing status based on sensor information 142 and/or patient information 144.

In some embodiments, healing information 146 may include information corresponding sensor information 142 with patient information 144 and a healing stage. For example, 500-1000 steps to achieve a 0.5-1.0 coulomb charge on capacitor 220 may be associated with a healing status of stage 2, 500-1000 steps to achieve a 1.5-2.5 coulomb charge on capacitor 220 may be associated with a healing status of stage 3, and so on. In another example, 500-1000 steps to charge an RF link may be associated with a healing status of stages 1 and/or 2, 1000-3000 steps to charge the RF link may be associated with a healing status of stage 3, and so on. In a further example, 500 steps to charge N channels of FG device may be associated with a healing status of stage 2, 1000 steps to achieve an injection rate of X of FG device may be associated with a healing status of stage 3, and so on.

In another example, healing information 146 may include a database, lookup table, or other data structure correlating step count information to expected sensor information values (for instance, stage 1: 500 Steps=capacitor charge of 1.0 coulombs; telemetry charge status of not charged; charge stored on the FG circuit of 1.0 V, etc.; stage 1: 1000 Steps=capacitor charge of 2.0 coulombs; telemetry charge status of charged; charge stored on the FG circuit of 2.0 V, etc.; stage 2: 500 Steps=capacitor charge of 0.5 coulombs; telemetry charge status of not charged; charge stored on the FG circuit of 0.5 V, etc.; stage 2: 1000 Steps=capacitor charge of 1.0 coulombs; telemetry charge status of not charged; charge stored on the FG circuit of 1.0 V, etc., and so on).

For example, with respect to telemetry charge information, healing information 146 may indicate that telemetry device 250 should be charged with 500 steps during a first healing stage, 1000 steps during a second healing stage, 2000 steps during a third healing stage, and so on. In another example, with respect to capacitor information, healing information 146 may indicate that 500 steps is expected to generate 1.0 coulomb of charge in capacitor 220 during a first healing stage, 1000 steps is expected to generate 2.0 coulombs of charge in capacitor 220 during the first healing stage, 500 steps is expected to generate 2.0 coulombs of charge in capacitor 220 during a second healing stage, 1000 steps is expected to generate 3.0 coulomb of charge in capacitor 220 during the second healing stage, and so on. In various embodiments, healing information 146 corresponding step count information to expected sensor information values may be based on experimental and/or estimated values based on population information. For example, healing information 146 associating, corresponding, matching, or otherwise linking step count values to expected sensor information values for patient 150 may be determined based on information determined based on a population of patients (including a corresponding population based on various correspondence factors, such as age, gender, weight, height, injury, surgery, and/or the like).

Healing status application 148 may apply any conversion factors and/or lookup the number of steps and compare the corresponding sensor information to determine a healing status. For instance, using the above example information, if the step count is 500 steps and the capacitor charge is 2.0 coulombs, healing status application 148 may determine that patient 150 is estimated to be in the second healing stage. In another instance, if the step count is 500 steps and the capacitor charge is 1.5 coulombs, healing status application 148 may determine a healing status that patient 150 is in between the first healing stage and the second healing stage. Embodiments are not limited in this context.

In some embodiments, the sensor charge information (i.e., charge information directly from strain sensor 162), telemetry charge information, capacitor information, and/or FG information may be used in combination, for example, to improve accuracy and/or verify a healing status determination. For example, capacitor information may indicate a healing status of stage 2. Healing status application 148 may determine the healing status based on FG information to determine whether this information also indicates a healing status of stage 2. In a further example, healing status application 148 may determine from direct measurement a charge generated by strain sensor 162 to determine whether this information also indicates a healing status of stage 2. In various embodiments, healing status application 148 may provide a healing status confidence score indicating a confidence of the determined healing status. The confidence score may be based on an amount of information used to determine the healing status (e.g., higher amounts information and/or longer duration of information provide higher confidence in the healing status) and/or confirmation between sensor information types. In another example, other patient information besides step count may be used, such as patient temperature (e.g., a higher temperature indicating an infection). Embodiments are not limited in this context.

In some embodiments, healing status application 148, for example, via healing status analysis logic 130, may lookup the number of steps in healing information 146 to determine an expected value for one or more types of sensor information 142 (i.e., capacitor information, FG information, and/or telemetry charge information). Healing status application 148 may compare the actual measured value of sensor information 142 to the expected values for each healing status or stage to determine the corresponding healing status (i.e., healing stage). Healing status application 148 may generate a healing status, for example, for presentation via display device 182.

In an example, healing status application 148 may receive a step count (for example, N steps) and access capacitor information indicating charge on capacitor 220. Healing information 146 may indicate an expected charge on capacitor for N steps for patient 150 for stages 1-4 (for instance, 0 coulombs for stage 1, X coulombs for stage 2, Y coulombs for stage 3, and Z coulombs for stage 4). Healing status application 148 may lookup N steps in healing information 146 and determine the matching stage for the capacitor charge. For example, if the capacitor charge is 0, then the healing status may be Stage 1, if the capacitor charge is X, then the healing status may be Stage 2, and so on.

In some embodiments, a healing information 146 may include a diagnosis generated by healing status application 148. For example, healing information 146 may include an expected healing stage for patient 150 (for instance, by week X, patient 150 is expected to be in Stage Y). When determining a healing status for patient 150, healing status application 148 may additionally determine a diagnosis or treatment recommendation, such as an indicator of the healing progress of patient, such as being on schedule, being ahead of schedule, being behind schedule, and/or the like. In some embodiments, the diagnosis or treatment recommendation may prompt patient to contact a healthcare provider if healing information 146 indicates an abnormal healing process or other serious issue with the progress of healing (for example, responsive to a patient being a stage behind an expected healing schedule).

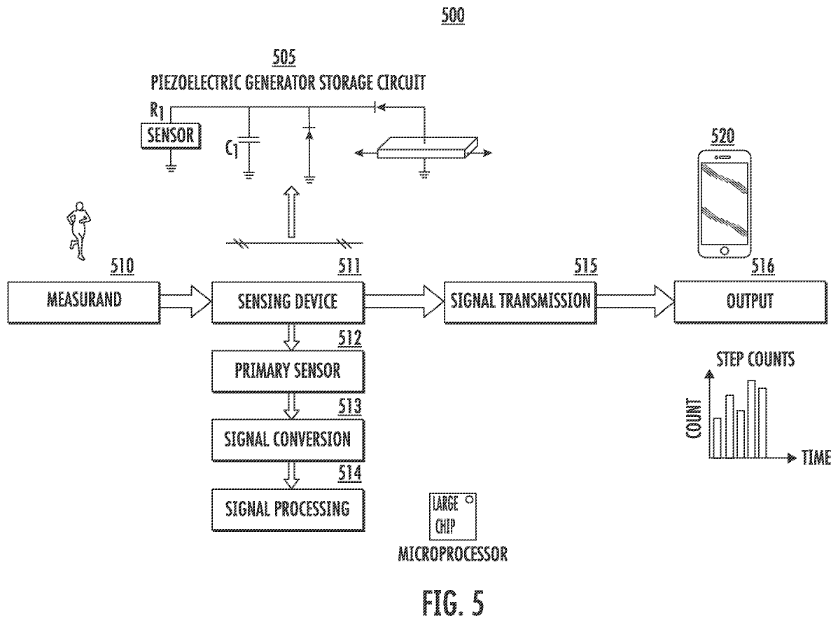

FIG. 5 depicts an example of a third operating environment that may be representative of some embodiments of the present disclosure. In general, FIG. 5 depicts an operating environment 500 of a system architecture for correlating step counts, for example, with energy storage in a capacitor according to some embodiments. Mechanical loads exerted onto the bone fixation device during rehabilitation is converted into a step count output from the energy stored on the capacitor. The charge signal generated by the PVDF sensor, which corresponds to the amount of strain measured by the sensor element, can then be directly measured by discrete signal monitoring via a wireless connection between the removable cap and an external reader.

As shown in FIG. 5, operating environment 500 may include a sensing device 511 that may include, for example, a self-powering, energy-harvesting piezoelectric generator storage circuit 505. Sensing device 511 may generate information (for instance, a charge) responsive to a measureand 510, such as steps or strain, measured by a primary sensor 512 (for instance, strain sensor 162). In various embodiments, circuits or other devices may be used for signal conversion 513 and/or signal processing. A transmitter may provide signal transmission 515 to provide output 516 at a computing device 520. For example, output 516 may be in the form of a healing status presented on a graphical user interface (GUI) presented via a screen of computing device 520.

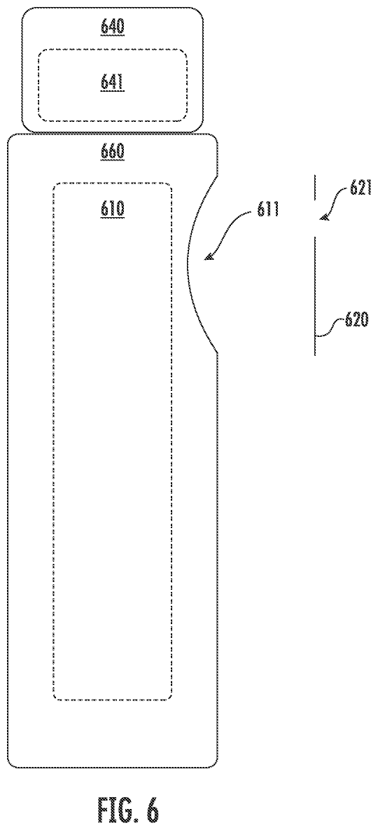

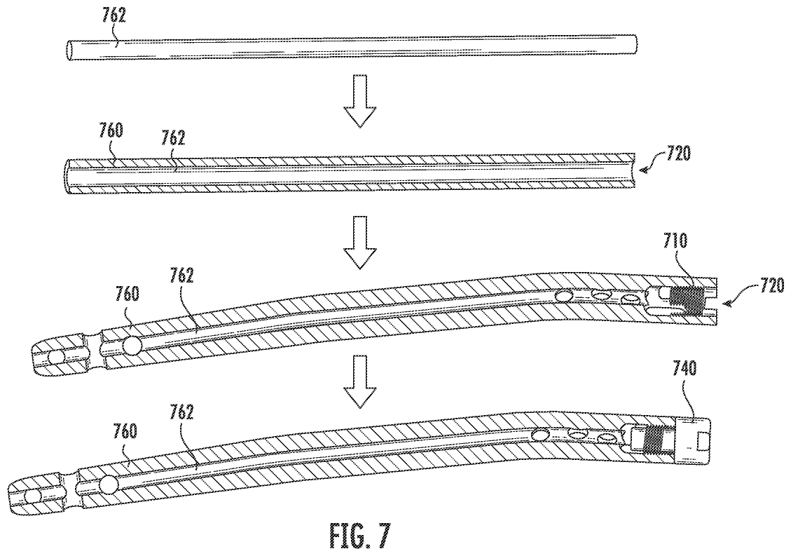

FIG. 6 depicts an example of a medical implant device having a plurality of mounting elements that may be representative of some embodiments of the present disclosure. As shown in FIG. 6, a medical implant device 660 may include one or more mounting elements for mounting a strain sensor, a telemetry device, batteries, power supplies, and/or other devices used according to some embodiments. In various embodiments, medical implant device 660 may include a cannulation 610 (internal to medical implant device 660 as indicated by the dashed lines of cannulation). For example, medical implant device 660 may be an IM nail having a central bore that is at least partially hollow. In some embodiments, strain sensor (and/or other devices) may be arranged within cannulation (see, for example, FIG. 7).

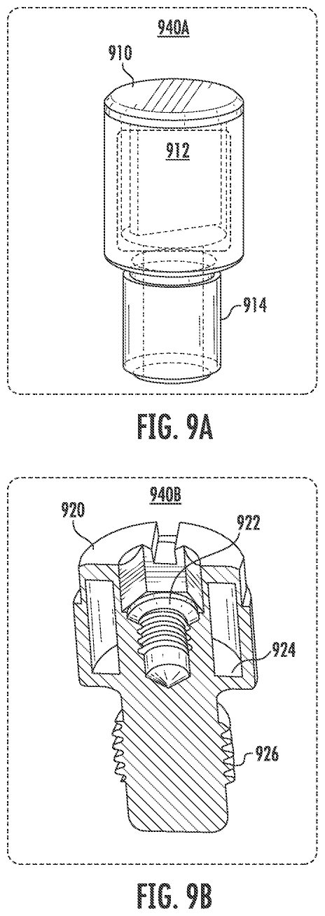

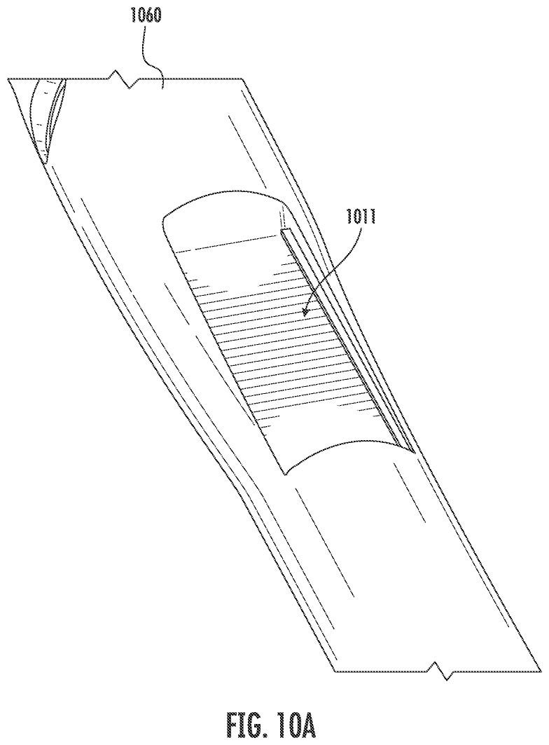

In various embodiments, a cap mounting device 640 may be configured to be coupled to medical implant device 660 (see, for example, FIGS. 9A and 9B). In various embodiments, cap 640 may include one or more storage cavities 641 for storing devices. In some embodiments, cap 640 may be used to store FG circuitry, a telemetry device, and/or a secondary power supply. In exemplary embodiments, an external cavity or pocket 611 may be machined or otherwise formed in an external surface of medical implant device 660 (see, for example, FIGS. 10A-10C). In various embodiments, at least a portion of a strain sensor may be arranged within pocket 611. For example, strain sensor may be or may include a foil material (e.g., PVDF) that may be mounted within pocket 611. In various embodiments, a lid 620 may be configured to at least partially seal pocket 611. In some embodiments, lid 620 may hermetically seal pocket. In exemplary embodiments, lid 620 may be formed of a material (for instance, titanium) and thickness (for instance, less than 100 microns and ideally 50 microns) capable of allowing a signal (for instance, a wireless signal) to be transmitted from pocket, for example, to a remote computing device. In various embodiments, lid 620 may include a window 621 formed of a transparent material that may allow a signal to be transmitted from pocket 611 through lid 620. In some embodiments, window 621 may be a ceramic window formed of an RF transparent material (for example, alumina, quartz, and/or zirconia). In various embodiments, lid 620 may be gold brazed to a titanium enclosure.

In various embodiments, strain sensor (for example, in the form of a film, such as PVDF film) may be bonded or otherwise affixed to surfaces of mounting elements of medical implant device 660 and/or directly to an external surface of medical implant device 660 using various depositing processes. In some embodiments, a PVDF sensor can be deposited onto the body of medical implant device 660 using a coating process, such as, for example, bar coating or spray coating adapted towards a tubular structure such as an IM nail, for instance, as described in Hiroki Takise et al. “Piezoelectric Vibration Energy Harvester Using Polyvinylidene Difluoride Film Formed by Bar Coating Method and Its Spray Coating Method on a Three Dimensional Surface,” Piezoelectricity, Chapter 5, Organic and Inorganic Materials and Applications (2018).

For bar coating, a PVDF solution can be prepared using methyl ethyl ketone (MEK), which is forced through a cannulation formed in the IM nail and the bar coater slid onto a spacer to expand the PVDF solution. On the surface of the bar-coater, grooves can be provided to obtain a uniform film thickness. The IM nail can be subsequently heated via, for example, a hot plate at 90° C. for 10 min to evaporate the MEK.

For spray coating, a spray nozzle can be set in a chamber facing down toward a work stage. The working part can be placed on a stage computationally controlled to move at a constant speed, e.g., 60 mm/s. The part can be fixed onto the stage by a vacuum chucking. The stage moves under the spray nozzle so as to draw a zigzag course repeatedly allowing uniform PVDF coating on the sample. The PVDF/MEK solution and nitrogen gas (for example, at a pressure of 0.4 MPa) can be supplied to a nozzle through individual tubes, and physically mixed, followed by sprayed in the form of circular cone having tip angle of 24°. The flow rate may be controlled at 60 g/h using a mass flow controller. A typical distance between the nozzle and work stage can be set to be 45 mm.

FIG. 7 depicts an example of a sensor device having cannulation mounting element that may be representative of some embodiments of the present disclosure. In some embodiments, a primary or strain sensor 762 may be pre-fabricated into a tube or substantially tubular shape and inserted into a cannulation 720 formed in a bone fixation device 760 (IM nail) during an early stage of the manufacturing process. In some embodiments, strain sensor 762 may be formed of PVDF. In some embodiments, strain sensor 762 may various thicknesses, for example, between about 50 to about 500 microns and can be pre-loaded onto a polymer/flexible core, which could be attached to a spool to allow it to be inserted into cannulation 720 of IM nail 760 and cut to length.

In various embodiments, dimensions of the core may be approximately about 300 mm long×5.4 mm outer diameter×5.2 mm inner diameter, which may correspond to the internal shape, length and diameter of an IM nail. In use, the size of a prefabricated PVDF tubing can be customized to fit any IM nail size in terms of length (e.g., between 18 and 50 cm), and internal diameter (e.g., 4.8 to 8.4 mm). The PVDF film preloaded onto a polymer core may then coated with a biocompatible adhesive and then pushed inside cannulation 720 of IM nail 760. In one embodiment, sensor tubing 762 may be bonded on the inside surface of cannulation 720 of an IM nail 760 using a biocompatible adhesive such as, for example, a USP Class VI epoxy adhesive (e.g., a 2-part or heat-curable epoxy from Master Bond of Hackensack, N.J., United States or Epoxy Technology Inc. of Billerica, Mass., United States). At this stage, PVDF sensor 762 may be inserted into IM nail 760 before the bow and bend operations are completed making the assembly easier. For example, transverse screw holes may be drilled in IM nail 760 and IM nail 760 may be bent.

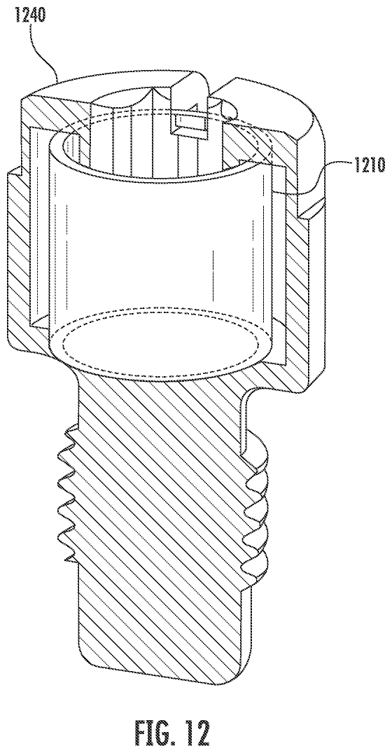

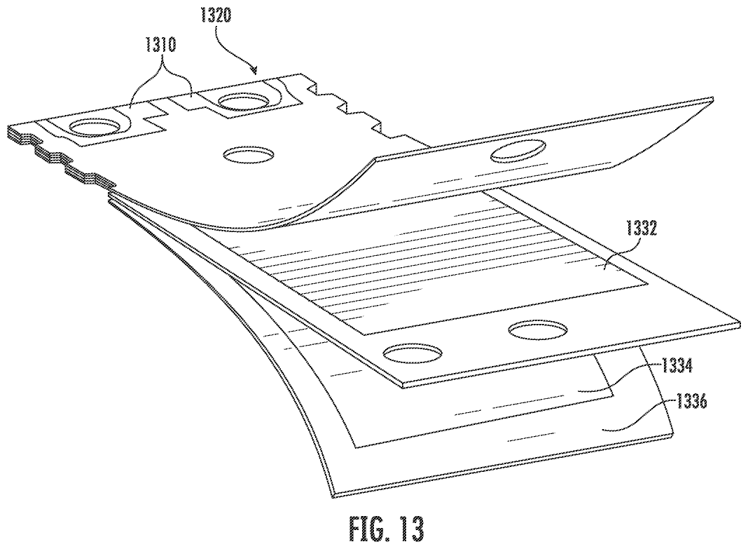

The polymer core can be removed leaving the PVDF film fixed to the inside surface of IM nail 760. The low profile design of the PVDF sensing system ensures that it will not interfere with existing surgical procedures for IM nailing such as deployment of the ball-tipped guide rod down the cannulation of IM nail 760 for controlling the action of the bone reamer and reducing the bone fragments. The pre-bent primary sensor tube may be seamlessly integrated within the IM nail and harvests its operational energy directly from mechanical activity of the device. In some embodiments, an instrumented cap 740 may be inserted into an end of IM nail 760 to make an electrical connection with strain sensor 762 (see, for example, FIGS. 13 and 14).

In one embodiment, the internal surface of IM nail 760 can be primed to improve the level of adhesion between IM nail 760 and primary sensor 762 using, for example, either a chemical (e.g., hydrogen peroxide, sulfuric acid, nitric acid, sodium hydroxide) or physical treatment (e.g., flame, plasma discharge, corona discharge, bead/sand blast, shot peen).

In one embodiment, a PVDF sensor may be flexible, stretchable, biocompatible, and/or incorporate a low profile nature of semi-crystalline plastic. Thus arranged, a PVDF sensor can be provided for circular-shaped devices such as IM nails for sensing changes in biomechanical loading. In one example of an embodiment, a PVDF sensor may be in the form of a film. In some embodiments, copolymers of PVDF such as PVDF tetrafluoroethylene (PVDF-TrFE) may be used that exhibit higher crystallinity due to their chemical structure, generally resulting in a better piezoelectric response.

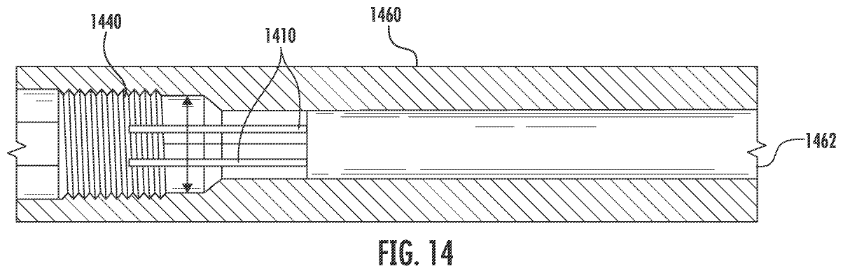

In use, output energy of the primary or strain sensor may be proportional to the volume of the film stressed. Film thickness may be chosen to optimize electrical signal and/or mechanical strength considerations. Thicker films generate higher voltages but form smaller capacitors. Any area of film that is not undergoing stress will act as a capacitive load on the “active” area and may be minimized, if required, according to some embodiments. Monomorphs or unimorphs are single layer films that are poled in only one direction that may produce maximum charge under uniaxial tension or compression; bimorph films are a two-layer construction in which each layer is poled in opposite directions so that maximum charge is produced in bending (see, for example, FIGS. 15-17).

In some embodiments, a PVDF strain sensor may include or may be formed from a thin metal layer deposited onto each side of the PVDF film. Thus arranged, two electrodes are created, and measurement of the charge generated is permitted. Possible manufacturing methods include screen printing with conductive silver ink (e.g., silver ink is best for applications where mechanical stress is being applied). The resulting metallization layer may be approximately 5-7 μm thick. Standard sputtered metallization may be 700 A of copper covered with 100 A of nickel, which exhibits good conductivity and is resistant to oxidation. Embodiments are not limited in this context.

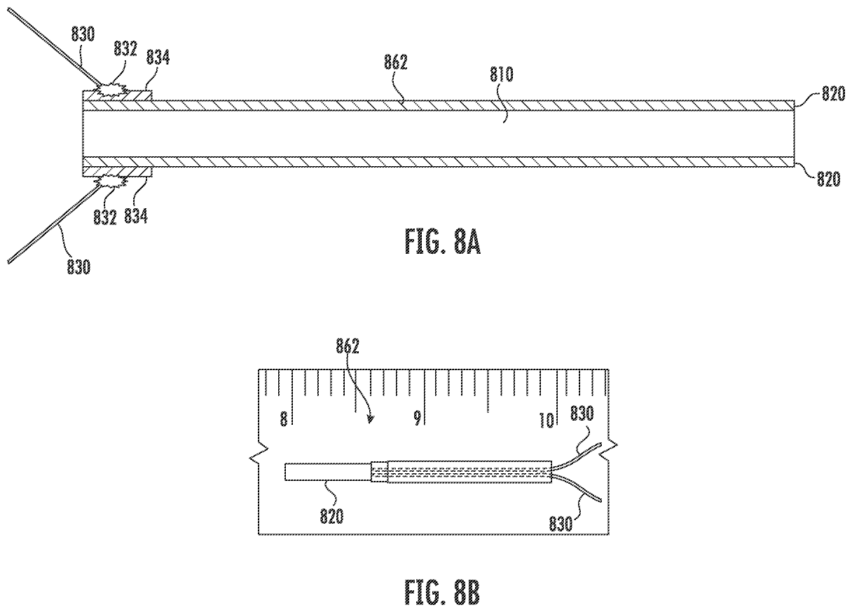

FIGS. 8A and 8B depict examples of medical implant device that may be representative of some embodiments of the present disclosure. As shown in FIGS. 8A and 8B, some embodiments may form an electrical connection with a PVDF sensor via wire lead attachments. For example, leads 830 may be attached to an electrode coating 820 to permit measurement of the current or voltage generated when the bone fixation device (e.g., IM nail) is subject to mechanical stress. In one embodiment, thin (for example, about 0.12 mm) copper (or another conductive metal) wires 830 with flexible electrical insulation may be used. Copper wires 830 may be bonded to PVDF sensor material 810 of strain sensor 862 using various techniques such as, for example, tape 834 (e.g., 3M® conductive copper tape provided by 3M Company of Saint Paul, Minn., United States) to affix wires 830 to a metallization intermediate layer 820 (for example, a silver ink metallization layer (electrode)). In use, copper tape 834 may operate to affix wires 830 to film 810 without damaging film 810 through the soldering process using solder 832 to attach wires 830 to tape 834 (e.g., since both film 810 and electrodes 830 are thin, the heat experienced during soldering directly to film 810 could damage or destroy both, which may be mitigated using copper tape 834).

FIGS. 9A and 9B depict examples of a cap mounting element that may be representative of some embodiments of the present disclosure. In some embodiments, a removable cap may be used in combination with a medical implant device. In various embodiments, a cap may be removably coupled to a medical implant device. For example, referring to FIG. 7, cap 740 may be removably coupled to one end of IM nail 760. In some embodiments, a cap may be used to mount or hold devices, such as strain sensors, telemetry devices, FG devices, circuitry, wires, and/or other components. In various embodiments, a cap can be used to establish a data measuring and transmission circuit connection between a PVDF sensor and an external reader unit. In one embodiment, cap 740 can be manufactured from non-metallic materials such as, for example, PEEK, PEEK composite, polyethylene, nylon, polypropylene polymer, variations thereof, combinations thereof, and/or the like to minimize wireless communication interference/attenuation (e.g., to reduce shielding to RF signals transmitted and received by the telemetry and external receiver unit).

Referring to FIG. 9A, a cap 940A may include a cavity 912 for holding devices or other components within cap 940A. Cavity 912, for example, a machined recess, may be enclosed via a lid 910. In some embodiments, lid 910 may create a hermetic seal protecting the implantable circuitry from biological fluid. Lid 910 may be attached to the body of cap 940A various means, such as ultrasonic or friction welding, an adhesive, a mechanical locking mechanism, friction fit, magnetic connector, and/or the like. A connector portion 914 may be configured to be coupled to a medical implant device. For example, connector portion 914 may be internally or externally threaded to engage with corresponding threads of a medical implant device. In another example, connector portion may be affixed to a medical implant device via various other means, such as an adhesive, welding, locking mechanism, friction fit, and/or the like. In some embodiments, cavity 912 may be configured to house an internal circuit board.

Referring to FIG. 9B, a cap 940B may utilize a threaded connection 926 to couple to an IM nail and a threaded connection 922 to couple to a driver, such as an external HEX driver, although other connection mechanisms are possible such as, for example, a custom design that utilizes an alternative means of connection, such as via a magnetic element (for instance, a magnetic strip). Cap 940B may include a welded lid 920 and a recess 924, such as a circular-shaped machined recess (for instance, having a size of 8 mm×2 mm) that may be used to house an antenna, telemetry device, wires, or other ancillary electronics.

In some embodiments, caps the same or similar to cap 940A or 940B may be used to store or mount components such as strain sensors, signal processing devices, such as analogue-to-digital converters (ADCs), one or more micro-controller units (MCUs), random-access memory (RAM), a secondary energy source such as a battery cell for continuous monitoring and data storage (avoiding blackouts), and an antenna. The antenna could be in the form of a flexible circuit, a coiled ferrite, or the like. Alternatively, the antenna could be housed in the cannulation of an IM nail.

FIGS. 10A-10C depict examples of an external cavity mounting element that may be representative of some embodiments of the present disclosure. As shown in FIG. 10A, a medical implant device (IM nail) 1060 may have a cavity or pocket 1011 formed on an external surface thereof. Referring to FIGS. 10B and 10C, an enclosure or lid 1050 may be configured to seal pocket 1011. In various embodiments, lid 1050 may include a window 1051 formed of a transparent or semi-transparent material. In some embodiments, lid 1050 may operate as a hermetic enclosure for pocket 1011.

In some embodiments, a thin film PVDF sensor may be bonded or otherwise affixed within recessed, machined pocket 1011 (e.g., 0.25-0.5 mm deep×100 mm long pocket formed in the device). In some embodiments, pocket 1011 may have a depth of about 0.1 mm, about 0.2 mm, about 0.25 mm, about 0.3 mm, about 0.4 mm, about 0.5 mm, about 0.75 mm, about 1.0 mm, or any value or range between any two of these values (including endpoints). In various embodiments, pocket 1011 may have a length of about 10 mm, about 20 mm, about 30 mm, about 40 mm, about 50 mm, about 100 mm, about 200 mm, about 300 mm, about 500 mm, a length the same or substantially similar to a length of IM nail 1060, or any value or range between any two of these values (including endpoints).

In some embodiments, lid 1050 may be formed of titanium, alloys thereof, and/or the like and may include an integrated RF transparent ceramic window for efficient data. transmission 1051. In some embodiments, window 1051 may be formed of a RF transparent or semi-transparent material including, without limitation, zirconia, quartz, or alumina, which, in various embodiments, may be brazed in situ or alternatively in a suitable titanium frame/housing that is subsequently welded to the body of the implant in a suitable inert atmosphere to guarantee a fully hermetic seal and corrosion free weld joint. In exemplary embodiments, lid 1050 may be placed over pocket 1011 and the mating surfaces may be welded to create a hermetic, watertight seal using suitable filling materials, e.g. covered electrodes, bare electrode wire or rod, tubular electrode wire, and welding fluxes, to fill any gaps that may arise between lid and implant and reduce the incidence of cracking as the weld cools.

In other embodiments, lid 1050 may not include a window. For example, lid may be formed of a material, such as titanium foil, which is thin enough (<100 μm) to allow wireless (e.g., RF) signals to pass through the metal material, for example, to energise an on-board antenna and allow data to be transmitted to and from a medical implant device to an external computing device.

FIG. 11 depicts a first example of a secondary power supply that may be representative of some embodiments of the present disclosure. In some embodiments, one or more secondary power sources may be associated with a medical implant device, for example, to power a telemetry device, antennae, signal processing devices, and/or the like. As shown in FIG. 11, a secondary power supply 1110 may include a inductive charger/transmitter potted, for example, in a medical grade silicone/epoxy resin. In some embodiments, secondary power supply 1110 may be located subcutaneously close to IM nail 1160 having strain sensor 1162 arranged therein. FIG. 12 depicts a second example of a secondary power supply that may be representative of some embodiments of the present disclosure.

As shown in FIG. 12, a secondary power supply 1210 may be arranged within a cap 1240 that may be coupled to an IM nail. The secondary power supply 1210 may be coupled to a strain sensor using various techniques. For example, a strain sensor arranged within the cannulation of a bone fixation device having a cap coupled to an end of the bone fixation device can be accomplished using, for example, conductive copper ring terminals (see, for example, FIG. 13) and/or a pair of lead wires (e.g., copper, silver, and/or gold) (see, for example, FIG. 14).

FIG. 13 depicts an example of a terminal ring electrical connection for a sensor device that may be representative of some embodiments of the present disclosure. As shown in FIG. 13, an electrical connection assembly may include a pair of terminal rings 1310. In some embodiments, terminal rings 1310 may be formed of a conductive metal, such as copper. The electrical connection assembly may include a piezo connection 1332, a copper (or other conductive metal) connection 1334, and an encapsulation layer 1336 (for instance, FR4). In some embodiments, electrical connection assembly may include mounting holes 1320, for example, for integration. FIG. 14 depicts an example of an electrical connection for connecting a sensor device to a device arranged within a cap mounting element that may be representative of some embodiments of the present disclosure. As shown in FIG. 14, a strain sensor 1462 of a medical implant device 1460 may be electrically coupled to one or more devices arranged in a cap 1440 via one or more lead wires 1410.

Piezoelectric actuators such as polyvinyl fluoride (PVDF) film are “energy harvesting” transducers that convert electrical energy into a mechanical displacement or stress based on a piezoelectric effect. The piezo properties are highly dependent on the β-phase content and its characteristics. During its production process, PVDF undergoes a stretching process and the film has different properties if this stretching is conducted in one or two directions. Typically, PVDF is received in a roll with a unidirectional alignment. The β-phase can be induced by several techniques, such as mechanical stretching of the α-phase films at a suitable temperature. The α-phase is developed directly from the melt. The conversion of a into β-phase takes place when stretching at temperatures below 100° C., using a stretch ratio between 3 and 5.

In one particular embodiments, a self-powered PVDF sensor may be formed to be embedded in the outer wall of a bone fixation device, such as IM nail. The PVDF sensors may be configured such that sensor output could be modified or monitored as a result of variations in (a) film thickness, (b) film orientation, (c) film layer configuration and (d) laminating adhesive.

In some embodiments, strain sensor configurations may be optimized to maximize current and/or voltage output. The minimum output power requirements from the PVDF sensor required to power a FG circuit continuously with an on-board antenna for data transmission is approximately 750 nW (5V/150 nA) (range of about 2 V to about 10V). This is the level that may be provided by the PVDF sensor for the lower bound strain level. The voltage increases with higher input forces and the circuit may have a cut-off diode at around 12 Volts. FG circuits can operate at less than 1 μW, which is well below the 50 mW that is currently required for commercially available ultralow-power wireless sensor nodes. The FG circuit may sense, store/record and compute cumulative statistics of loading cycles experienced by medical implant devices according to some embodiments.

Regarding film thickness, the inherent capacitance of the film may, in combination with the associated electronics, affect sensor output. In some embodiments, sensor thickness may be between about 25 and about 52 microns depending on a particular configuration (see, for example, Table 1). The output energy is proportional to the volume of film stressed. Film thickness can be used to optimize the electrical signal and mechanical strength considerations. Films having a thickness greater than 52 microns generate higher voltages but form smaller capacitors.

Regarding film orientation, in some embodiments, film orientation may be uniaxial stretch. Uniaxial film has one strong lateral extension mode (d31) and one weak lateral mode (d32). Typically, the d31 mode is 9 times larger than d32 in uniaxial film. Bi-directional film has moderate lateral extension modes in both directions so that d31 and d32 are approximately equal. Furthermore, these are both approximately one third the value of d31 in uniaxial film but 3 times the value of d32 in uniaxial film. The thickness mode response d33 does not vary as a function of stretching method.

Regarding film configuration, in some embodiments, output sensitivity of the self-powered sensor may be maximized to bending/flexural modes, which typically occur when the IM nail is subjected to compressive, bending and torsional physiological loading conditions. Ligaments and tendons apply compression and torque to the bones depending on their location and direction of strain, while body weight induces compression on the bones; these loads are directly transmitted to the IM nail when implanted. In some embodiments, the dominant mode of stress of a medical device implant may be compressional depending on position of the patient and phase within the gait cycle. In other embodiments, the dominant mode of stress of a medical device implant may be flexural. In some embodiments, PVDF sensor film may have configurations varied from “uni-morph” to “series stack” and “bi-morph” (see, for example, Table 1).

FIGS. 15-17 depict schematic illustrations of examples of energy-harvesting polyvinyl fluoride (PVDF) film sensor devices that may be representative of some embodiments of the present disclosure. Referring to FIG. 15, therein is depicted a schematic illustration of a single/monolithic/uni-morph sensor 1562 arranged within electrodes 1510 (for example, evaporated gold electrodes) highlighting the higher sensitivity to thickness “compressional” mode, but a limited sensitivity to flexural mode. In FIGS. 15-17, arrows denote direction of polarizations. A “uni-morph” configuration has only one active/sensing layer (the piezoelectric layer) in the sensor bonded onto a non-piezoelectric layer (metal conductor of the generated charge).

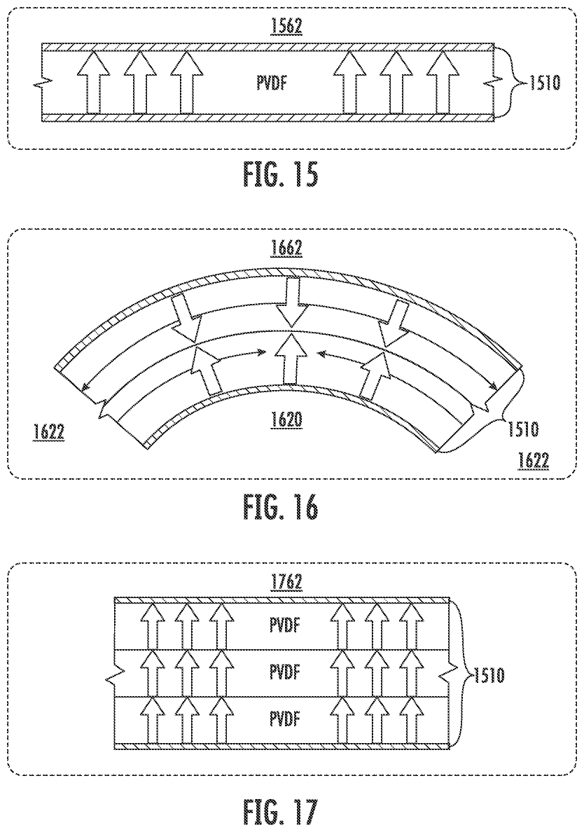

Referring to FIG. 16, therein is depicted a “bi-morph” sensor 1662 having layers 1610, with inverted polarizations and sensitive to compression 1620 and tension 1622. A bi-morph sensor configuration may have higher sensitivity to flexural mode but insensitivity to thickness “compressional” mode. In some embodiments, a “bi-morph” configuration may include bonding two thin, active layers of piezoelectric polymer onto the same metal layer to increase the power output. Bi-morph structures may double the energy output of an energy harvester without a significant increase in the device volume.

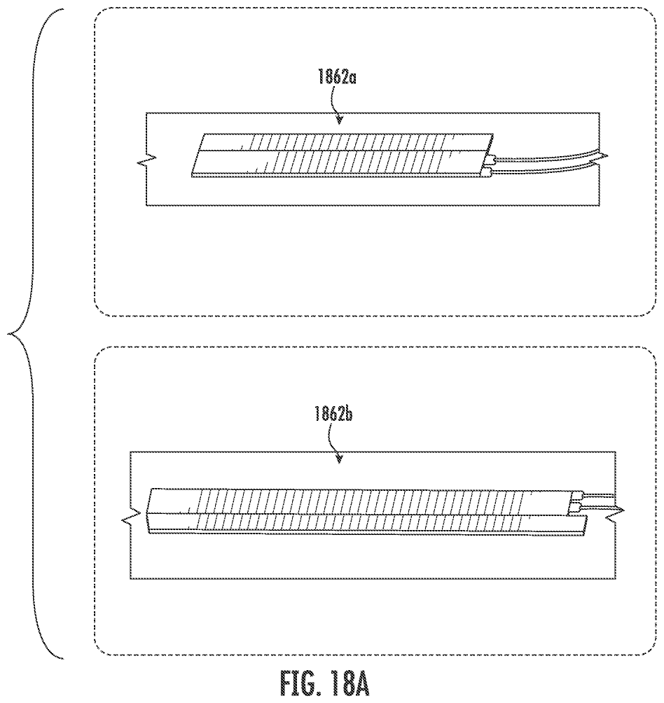

Referring to FIG. 17, therein is depicted a series stack configuration highlighting the higher sensitivity to thickness “compressional” mode but poor sensitivity to flexural mode. Where PVDF stacks are laminated together, the dielectric properties of the adhesive used, impacts the capacitive coupling and thus may also affect sensor output.

The following Table 1 provides non-limiting example embodiments of various sensor configurations:

TABLE 1

| PVDF | Adhesive | Configuration |
|---|---|---|
| 52 μm (uni-axial) | Non-medical grade adhesive | Monolithic |
| | | block |
| 52 μm (uni-axial) | Non-medical grade adhesive | Bi-morph |
| 52 μm (uni-axial) | Bio-compatible USP class VI epoxy | Series Stack |
| | | of 3 |
| 52 μm (uni-axial) | Bio-compatible USP class VI epoxy | Bi-morph |
| 28 μm (uni-axial) | Bio-compatible USP class VI epoxy | Bi-morph |
| 28 μm (uni-axial) | Bio-compatible USP class VI epoxy | Monolithic |
| | | block |
| 25 μm (bi-axial) | Bio-compatible USP class VI epoxy | Monolithic |
| | | block |
| 25 μm (bi-axial) | Bio-compatible USP class VI epoxy | Bi-morph |

### Examples: PVDF Sample Sensors

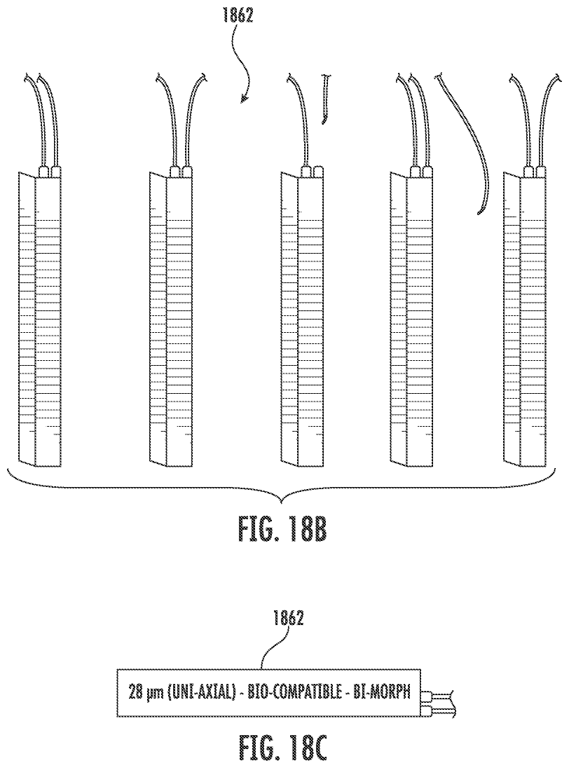

PVDF sample sensors were generated using processes according to some embodiments. The PVDF sample sensors included sensors/electrode assemblies that were metallized. For example, a sensor may consist of PVDF film with a gold outer conductive layer and a chrome inner “prime layer” to allow the gold to bond to the hydrophobic PVDF surface. FIG. 18A-18C depict sample sensors of energy-harvesting PVDF film sensor devices that may be representative of some embodiments of the present disclosure. Referring to FIG. 18A, therein is depicted sample sensors 1862a and 1862b in the form of rectangular-shaped laminated PVDF sensors. In some embodiments, gold/chrome electrodes are deposited onto the PVDF by either evaporation or sputtering using a suitable mask required for the sputter deposition of the electrode pattern.

Once laminated and associated with electrodes, sample sensors were bonded to titanium coupons and electrode wires were attached to the electrode tags. Electrode connections were reinforced with a flexible silicone adhesive to try and protect the integrity of the connection. The sample sensors were bonded to the pocket floor using a suitable adhesive, for example, EP30MED and EP42HT-2MED provided by Master Bond Inc. of Hackensack, N.J., United States. Both adhesives are two component epoxy systems, which not only meet the USP Class VI requirements for biocompatibility, but have been tested for the International Standards Organization (ISO) 10993-5 non-cytotoxicity requirements. The surfaces are thoroughly cleaned and roughened prior to application to help improve the bond strength between the PVDF sensor and underlying metal surface. The ideal cure schedule for both would be an overnight room temperature set up, followed by an addition of heat to about 150° F. or higher for around 3 to 5 hours.

FIGS. 18B and 18C depict front and back views, respectively, of rectangular-shaped PVDF sample sensors 1862 bonded to a block of titanium-64 with a surface area of 8×70 mm mirroring the footprint of a machined pocket in a bone fixation device. Sensors 1862 are comprised of sputter coated gold/chrome electrodes on the top and bottom surfaces with bonded flying leads. The input impedance of the circuit at the PVDF pins may vary from 70 to 30 MegaOhms.

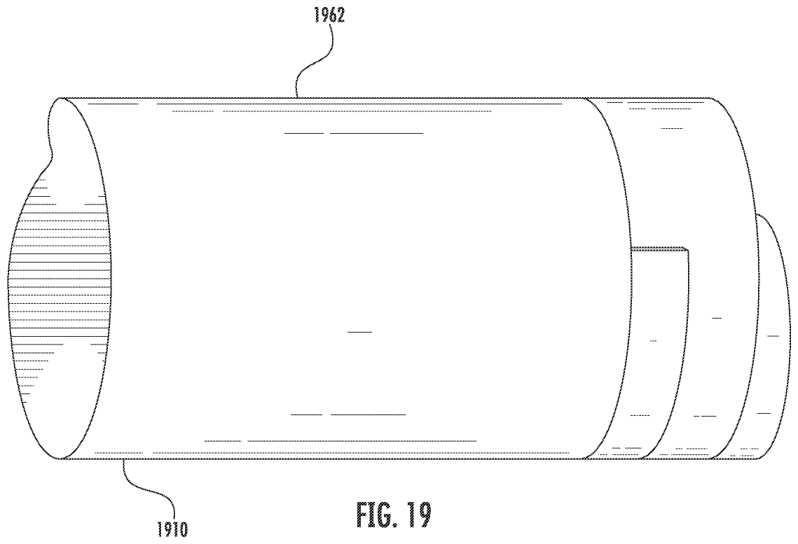

FIG. 19 depicts an example of a cylindrical sensor that may be representative of some embodiments of the present disclosure. As shown in FIG. 19, in an alternative embodiment, a PVDF sample sensor 1962 was arranged in a cylindrical shaped form to allow it to be attached to the inside surface of the cannulation of a bone fixation device. Cylindrically shaped PVDF sensor 1962 was formed of sputter coated gold/chrome electrodes 1910. The sensor is designed to be inserted into the cannulation of the IM nail

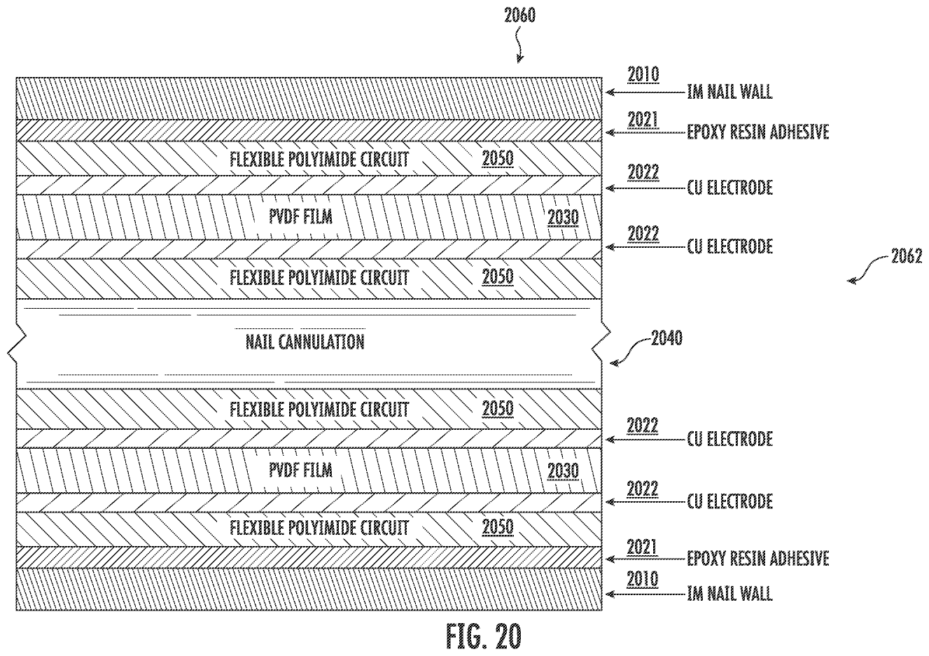

FIG. 20 depicts a cross-sectional (along a longitudinal axis) side view of a portion of a cannulated medical implant device that may be representative of some embodiments of the present disclosure. As shown in FIG. 20, a medical implant device 2060 (e.g., IM nail) may include a cylindrical sensor 2062 arranged within cannulation 2040 that ultimately form multiple layers within IM nail wall 2010. Non-limiting examples of layers may include epoxy resin adhesive 2021, a flexible polyimide circuit 2040, an electrode 2022 (for instance, a copper electrode), and a PVDF film 2030.

The flexible polyimide circuit may be about 120 microns thick. The overall wall section of the sensor may be about 0.5 mm, which is thin enough to be inserted within the cannulation of an intramedullary nail without compromising the ability to insert surgical instruments, e.g., ball tip guide rods and/or the like. The length of the PVDF sensor tubing may be restricted to the distance between the inferior proximal screw and the most superior distal screw, for instance, to avoid any local perforations/discontinuities in the films to accommodate the interlocking screws that may degrade sensor performance.

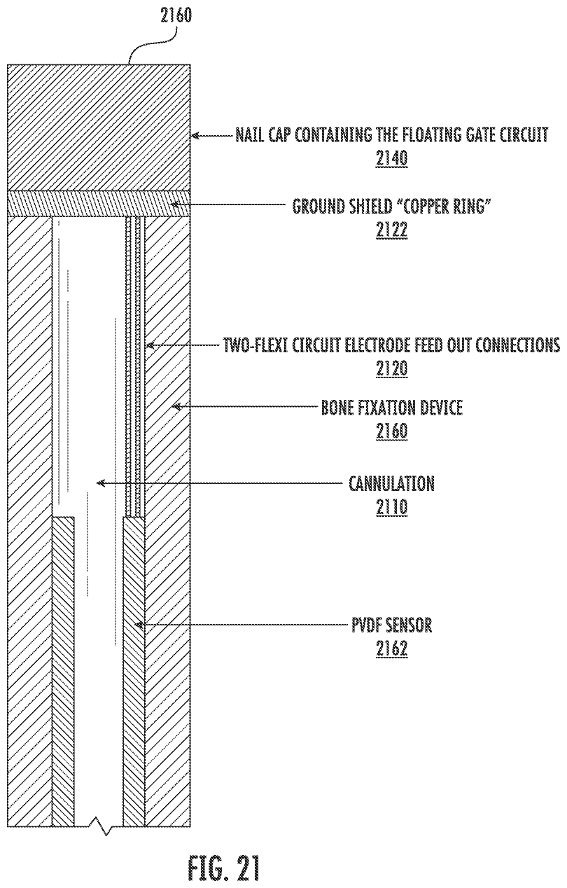

FIG. 21 depicts a cross-sectional side view of a medical implant device having a cylindrical sensor that may be representative of some embodiments of the present disclosure. As shown in FIG. 21, a medical implant device 2160 (IM nail) may include a cylindrical PDF sensor 2162. In some embodiments, medical implant device 2160 may include or may be associated with a cap 2140 (for example, housing a FG circuit or other device), a ground shield 2122 (for instance, implemented via a metal ring, such as a copper ring), electrode feed out connections 2120 (for instance, two flexi-circuit electrode feed out connections). Sensor 2162 may be arranged with a cannulation 2110 of IM nail 2160.

Electrical contact between cylindrical-shaped PVDF sensor 2162 and an FG circuit located in cap 2140 at the proximal end of IM nail 2160 may be implemented using ground shield 2122 located in a key-way section at the proximal end of IM nail 2160.

Included herein are one or more logic flows representative of exemplary methodologies for performing novel aspects of the disclosed architecture. While, for purposes of simplicity of explanation, the one or more methodologies shown herein are shown and described as a series of acts, those skilled in the art will understand and appreciate that the methodologies are not limited by the order of acts. Some acts may, in accordance therewith, occur in a different order and/or concurrently with other acts from that shown and described herein. For example, those skilled in the art will understand and appreciate that a methodology could alternatively be represented as a series of interrelated states or events, such as in a state diagram. Moreover, not all acts illustrated in a methodology may be required for a novel implementation.

A logic flow may be implemented in software, firmware, hardware, or any combination thereof. In software and firmware embodiments, a logic flow may be implemented by computer executable instructions stored on a non-transitory computer readable medium or machine readable medium. The embodiments are not limited in this context.

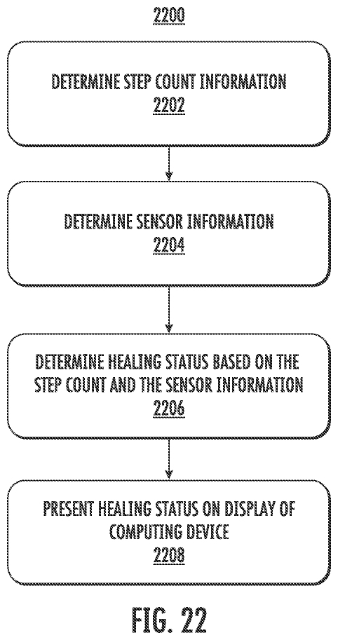

FIG. 22 illustrates an embodiment of a logic flow 2200. Logic flow 2200 may be representative of some or all of the operations executed by one or more embodiments described herein, such as computing device 110. In some embodiments, logic flow 2200 may be representative of some or all of the operations of a healing status analysis process according to some embodiments.

At block 2202, logic flow 2200 may determine step count information. For example, healing status application 148 may access patient information 144 measured by patient sensor 164 indicating a step count associated with patient 150, for instance, during a specific measurement period. Logic flow 2200 may determine sensor information at block 2204. For example, healing status application 148 may determine sensor information 142 in the form of capacitor information, telemetry charge information, and/or FG information during or at the end of the measurement period.

Logic flow 2200 may determine a healing status based on the step count and the sensor information. For example, healing information 146 may include a database, lookup table, or other data structure correlating step count information to expected sensor information values. For example, with respect to telemetry charge information, healing information 146 may indicate that telemetry device 250 should be charged with 500 steps during a first healing stage, 1000 steps during a second healing stage, 2000 steps during a third healing stage, and so on. In another example, with respect to capacitor information, healing information 146 may indicate that 500 steps is expected to generate 1.0 coulomb of charge in capacitor 220 during a first healing stage, 1000 steps is expected to generate 2.0 coulombs of charge in capacitor 220 during the first healing stage, 500 steps is expected to generate 2.0 coulombs of charge in capacitor 220 during a second healing stage, 1000 steps is expected to generate 3.0 coulomb of charge in capacitor 220 during the second healing stage, and so on.

Healing status application 148 may apply any conversion factors and/or lookup the number of steps and compare the corresponding sensor information to determine a healing status. For instance, using the above example information, if the step count is 500 steps and the capacitor charge is 2.0 coulombs, healing status application 148 may determine that patient 150 is estimated to be in the second healing stage. In another instance, if the step count is 500 steps and the capacitor charge is 1.5 coulombs, healing status application 148 may determine a healing status that patient 150 is in between the first healing stage and the second healing stage. Embodiments are not limited in this context. In some embodiments, healing status application 148 may determine whether patient 150 is experience an abnormal healing process, for instance, because healing status application 148 has determined that patient 150 is in a healing stage behind an expected healing stage and/or based on other physiological information of patient 150 (e.g., temperature, weight, gait information, and/or the like).

At block 2208, logic flow 2200 may present the healing status on a display of a computing device. For example, healing information 146 in the form of a healing status (e.g., “Stage 1 of Fracture Healing”) may be presented on display 182 of computing device 110. In this manner, the patient and/or healthcare professional may monitor the healing progress of an effected area of the patient. In another example, healing status application 148 may generate an abnormal healing status message responsive to a determination of abnormal healing. In some embodiments, the displayed healing status may include a treatment recommendation, for example, for a healthcare professional to examine a cause of the abnormal healing, generated based on the sensor information 142, patient information 144, and/or healing information 146.

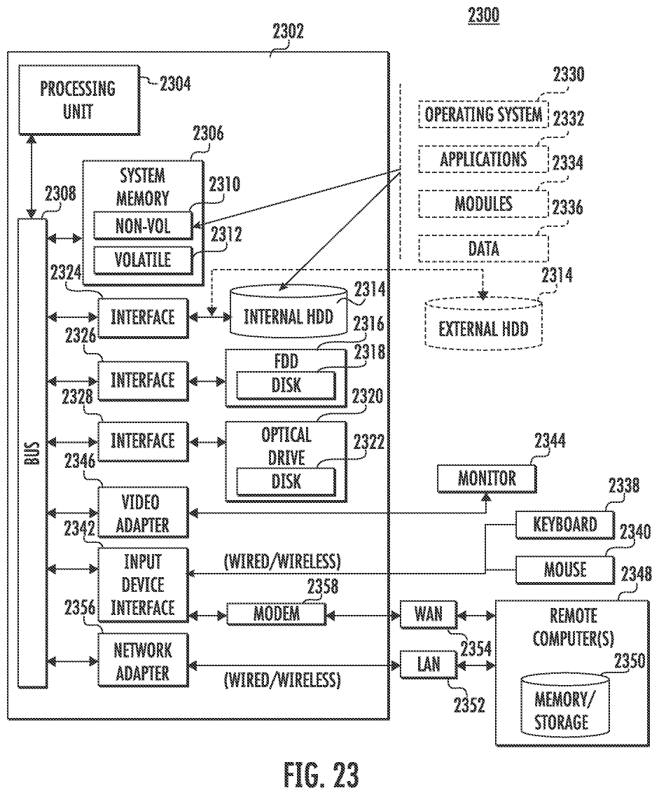

FIG. 23 illustrates an embodiment of an exemplary computing architecture 2300 suitable for implementing various embodiments as previously described. In various embodiments, the computing architecture 2300 may comprise or be implemented as part of an electronic device. In some embodiments, the computing architecture 2300 may be representative, for example, of computing device 110. The embodiments are not limited in this context.

As used in this application, the terms “system” and “component” and “module” are intended to refer to a computer-related entity, either hardware, a combination of hardware and software, software, or software in execution, examples of which are provided by the exemplary computing architecture 2300. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers. Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

The computing architecture 2300 includes various common computing elements, such as one or more processors, multi-core processors, co-processors, memory units, chipsets, controllers, peripherals, interfaces, oscillators, timing devices, video cards, audio cards, multimedia input/output (I/O) components, power supplies, and so forth. The embodiments, however, are not limited to implementation by the computing architecture 2300.

As shown in FIG. 23, the computing architecture 2300 comprises a processing unit 2304, a system memory 2306 and a system bus 2308. The processing unit 2304 may be a commercially available processor and may include dual microprocessors, multi-core processors, and other multi-processor architectures.

The system bus 2308 provides an interface for system components including, but not limited to, the system memory 2306 to the processing unit 2304. The system bus 2308 can be any of several types of bus structure that may further interconnect to a memory bus (with or without a memory controller), a peripheral bus, and a local bus using any of a variety of commercially available bus architectures. Interface adapters may connect to the system bus 2308 via a slot architecture. Example slot architectures may include without limitation Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and the like.

The system memory 2306 may include various types of computer-readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In the illustrated embodiment shown in FIG. 23, the system memory 2306 can include non-volatile memory 2310 and/or volatile memory 2312. A basic input/output system (BIOS) can be stored in the non-volatile memory 2310.

The computer 2302 may include various types of computer-readable storage media in the form of one or more lower speed memory units, including an internal (or external) hard disk drive (HDD) 2314, a magnetic floppy disk drive (FDD) 2316 to read from or write to a removable magnetic disk 2311, and an optical disk drive 2320 to read from or write to a removable optical disk 2322 (e.g., a CD-ROM or DVD). The HDD 2314, FDD 2316 and optical disk drive 2320 can be connected to the system bus 2308 by a HDD interface 2324, an FDD interface 2326 and an optical drive interface 2328, respectively. The HDD interface 2324 for external drive implementations can include at least one or both of Universal Serial Bus (USB) and IEEE 1114 interface technologies.

The drives and associated computer-readable media provide volatile and/or nonvolatile storage of data, data structures, computer-executable instructions, and so forth. For example, a number of program modules can be stored in the drives and memory units 2310, 2312, including an operating system 2330, one or more application programs 2332, other program modules 2334, and program data 2336. In one embodiment, the one or more application programs 2332, other program modules 2334, and program data 2336 can include, for example, the various applications and/or components of computing device 110.

A user can enter commands and information into the computer 2302 through one or more wired/wireless input devices, for example, a keyboard 2338 and a pointing device, such as a mouse 2340. These and other input devices are often connected to the processing unit 2304 through an input device interface 2342 that is coupled to the system bus 2308, but can be connected by other interfaces.

A monitor 2344 or other type of display device is also connected to the system bus 2308 via an interface, such as a video adaptor 2346. The monitor 2344 may be internal or external to the computer 2302. In addition to the monitor 2344, a computer typically includes other peripheral output devices, such as speakers, printers, and so forth.

The computer 2302 may operate in a networked environment using logical connections via wired and/or wireless communications to one or more remote computers, such as a remote computer 2348. The remote computer 2348 can be a workstation, a server computer, a router, a personal computer, portable computer, microprocessor-based entertainment appliance, a peer device or other common network node, and typically includes many or all of the elements described relative to the computer 2302, although, for purposes of brevity, only a memory/storage device 2350 is illustrated. The logical connections depicted include wired/wireless connectivity to a local area network (LAN) 2352 and/or larger networks, for example, a wide area network (WAN) 2354. Such LAN and WAN networking environments are commonplace in offices and companies, and facilitate enterprise-wide computer networks, such as intranets, all of which may connect to a global communications network, for example, the Internet.

The computer 2302 is operable to communicate with wired and wireless devices or entities using the IEEE 802 family of standards, such as wireless devices operatively disposed in wireless communication (e.g., IEEE 802.16 over-the-air modulation techniques). This includes at least Wi-Fi (or Wireless Fidelity), WiMax, and Bluetooth™ wireless technologies, among others. Thus, the communication can be a predefined structure as with a conventional network or simply an ad hoc communication between at least two devices. Wi-Fi networks use radio technologies called IEEE 802.11x (a, b, g, n, etc.) to provide secure, reliable, fast wireless connectivity. A Wi-Fi network can be used to connect computers to each other, to the Internet, and to wire networks (which use IEEE 802.3-related media and functions).

In accordance with one or more features of the present disclosure, continuous battery-less monitoring of bone fixation devices can be achieved using a self-powered sensor and an integrated circuit. For example, the system could utilize less than 1 uW of power. In one embodiment, floating-gate transistors allow piezoelectric materials such as PVDF to remain operational for several months at this level of power consumption. In one approach, the progression of fracture healing can be established by the amount of energy stored on a capacitor and the number of steps used to power a simple RF link. The number of steps taken by the patient during rehabilitation, which are required to power the RF link, could directly indicate the magnitude of the mechanical force carried by the bone fixation device. As normal healing progresses, the bone carries more mechanical load and thus the number of steps to power the RF link increases. In the event of any aberrant healing, when either the implant fails or bone fails to unite, the reduction in step count that occurs can be used by the healing status application and/or care-provider to determine a treatment recommendation, such as to plan a salvage/revision procedure. In a second approach, the progression of fracture healing can be determined from the stored cumulative statistics of the strain rates experienced by the bone fixation device through the use of digital floating gate circuitry, which can be remotely retrieved using RFID technology. The ultra-low voltage floating gate transistor circuit allows the primary sensor to (a) be powered at <1 μW, (b) support automatic data collection/transmission and computation of cumulative stress and strain patterns exerted on the bone fixation device using an ultra-low-power, long-range wireless interface. and (c) can be packaged within a small footprint that fits within a removable nail cap.

In one embodiment, as previously described, the PVDF sensor is used to correlate the amount of stress placed on a bone fixation device with an amount of power signal corresponding transmitted to an external receiver via a telemetry unit. In addition, and/or alternatively, other sensing devices can be incorporated. For example, one or more sensing devices may be incorporated into the nail cap. For example, a thermistor or an inductive capacitive (LC) circuit for monitoring temperature at the site of implantation based on the hypothesis that infection can cause local temperature to rise around the bone fixation device, an accelerometer for counting steps to determine patient activity, etc.

It should be noted that it is envisioned that the sensor (e.g., PVDF sensor) could be coupled to the bone fixation device in other manners. For example, in one embodiment, the sensor could be threaded into a corresponding threaded portion of the bone fixation device. The sensor could have a ring of polyethylene that mates with a corresponding intermittent circumferential grove. The sensor could be made primarily and/or entirely from polyethylene and incorporate the locking ring. The sensor cold be over-molded with polyethylene or any other suitable biocompatible material. In addition, and/or alternatively, the sensor could incorporate a cannulation to facilitate its removal should the bone fixation device need to be removed.

In one embodiment, a bone fixation device such as, for example, an intramedullary (IM) nail, comprising at least one implantable self-powered sensor for the purpose of monitoring an aspect of patient health such as, for example, implant strain, is disclosed.

In another embodiment, a bone fixation device comprising a primary sensor that spans an entire length of bone fixation device is disclosed. Thus arranged, the need for multiple sensors deployed along the length of the bone fixation device to ensure that at least one sensor is always located at or close to the fracture site for maximum sensitivity for detecting load changes on the bone fixation device during fracture healing is mitigated.

In another embodiment, a bone fixation device such as, for example, an intramedullary (IM) nail, comprising a cannulation and a self-powered sensor that is tailored in size to the cannulation. The bone fixation device may be made from either a polyvinylidene difluoride (PVDF) or one its co-polymers for the purpose of converting mechanical energy into a measurable charge.

In another embodiment, a bone fixation device comprising a cannulation, which can be used for accessing surgical instruments and for housing a primary sensor in the form of a tube or a coating is disclosed.

In one embodiment, a method for attaching a primary sensor to a portion(s) of an inside surface of a bone fixation device that can withstand the forces typically experienced during surgical implantation is disclosed.

In another embodiment, a method for inserting a primary sensor into a portion of a cannulation in the form of a plug before or after shape forming operations is disclosed. Plug insertion could occur during surgery to implant the bone fixation device. In one embodiment, removal of the primary sensor is also preferably enabled.

In another embodiment, a method for applying a primary sensor to a tubular internal surface of a bone fixation device whereby the primary sensor is a thin tube with an inner cylindrical flexible polymeric rod to facilitate insertion of the primary sensor into the bone fixation device is disclosed. In one embodiment, the primary sensor is inserted before the bone fixation device is bent and/or bowed. In one embodiment, the primary sensor is inserted before locking screw holes are added to the bone fixation device. In one embodiment, the cylindrical, flexible polymeric rod could be expandable to allow for application/adhesion of the primary sensor to the inner surface of the bone fixation device. In one embodiment, the flexible, cylindrical, polymeric rod is removable once the primary sensor is fixed to the bone fixation device.

In another embodiment, a method for priming an internal surface of a bone fixation device for improving the level of adhesion between the bone fixation device and a primary sensor is disclosed. Priming the internal surface can be by either a chemical such as, for example, hydrogen peroxide, sulphuric acid, nitric acid, sodium hydroxide, or the like, or a physical treatment such as, for example, flame, plasma discharge, corona discharge, bead/sand blast, shot peen, or the like.

Alternatively, in another embodiment, a method of bonding a primary sensor to an internal surface of a bone fixation device using either a medical grade adhesive such as an epoxy resin or using other methods such as contact welding, radiant heat, RF induction, mechanical means (e.g., press-fit, dove-tail, etc.) is disclosed.

In another embodiment, a bone fixation device that connects to a removable cap via, for example, a thread, pin or magnetic strip, which contains an integrated circuit that is capable of integrating the energy harvested from the primary sensor with the data collection/conversion/storage utilities through the use of a floating gate transistor circuit is disclosed.

In another embodiment, a removable cap comprising a secondary power source in the absence of a floating gate circuit to allow continuous monitoring during bone healing is disclosed.

In another embodiment, a removable cap comprising a transmitter to allow wireless power and data transmission to determine the progress of fracture healing is disclosed.

In another embodiment, a removable cap made from a non-metallic material such as PEEK, PEEK composite, polyethylene or nylon to reduce the impact of shielding to RF signals transmitted and received by the telemetry and external receiver unit is disclosed.

In another embodiment, a method for establishing electrical connection between a piezoelectric sensor and an electronic circuitry of a telemetry unit housed in a remote screw cap attached to an end of a bone fixation device using a biologically inert metallic material such as gold, copper or silver in the form of a wire or ring terminal is disclosed.

In another embodiment, a method for attaching a bone fixation device to an instrumented nail cap using either a thread or magnetic connector or pin is disclosed.

In another embodiment, a primary bone healing algorithm that can be inferred from the number of steps taken by a patient, which are required to power a wireless electromagnetic signal from the primary sensor that is exposed to a biomechanical load, to an external receiver unit and the amount of charge stored on the generator storage circuit is disclosed.

In another embodiment, a secondary bone healing algorithm that can be interpreted from a floating gate transistor injector whereby the sensor computes the cumulative strain patterns experienced by the bone fixation device during fracture healing without having to utilize data converters is disclosed.

In another embodiment, an external unit, which is capable of remotely processing and interpreting the number of steps and the change in capacitance from the amount of load exerted on the implant during healing, i.e. the load imposed upon the bone fixation device and the charge generated in response to the decreasing load, as bone healing progresses is disclosed.

In another embodiment, an external unit, which is capable of remotely processing the stress and strain statistics data obtained from the IM nail in response to the decreasing load, as bone healing progresses through the use of RFID technology is disclosed.

In another embodiment, an external unit that could be in the form of a handheld device such as mobile phone or a wearable sleeve or brace that could be worn by the patient during active hours to inductively power the implant and also include functionality for wireless transmission to a mobile phone or other wirelessly enable device is disclosed.

While the present disclosure has been illustrated and described in detail in the drawings and foregoing description, the same is to be considered as illustrative and not restrictive in character, it being understood that only the certain embodiments have been shown and described and that all changes, alternatives, modifications and equivalents that come within the spirit of the disclosure are desired to be protected.

It should be understood that while the use of words such as preferable, preferably, preferred or more preferred utilized in the description above indicate that the feature so described may be more desirable, it nonetheless may not be necessary and embodiments lacking the same may be contemplated as within the scope of the present disclosure, the scope being defined by the claims that follow. In reading the claims, it is intended that when words such as “a,” “an,” “at least one,” or “at least one portion” are used there is no intention to limit the claim to only one item unless specifically stated to the contrary in the claim. When the language “at least a portion” and/or “a portion” is used the item can include a portion and/or the entire item unless specifically stated to the contrary.

What is claimed is:

1. A medical implant device for attachment to a portion of a bone, comprising:
at least one energy-harvesting strain sensor operative to generate a charge responsive to a strain force on the medical implant device; and
at least one electronic element configured to receive at least a portion of the charge, the at least one electronic element comprising a telemetry device operably coupled to the at least one energy-harvesting sensor, the telemetry device powered, at least in part, by the at least a portion of the charge to transmit sensor information associated with the charge to a receiver device.

2. The medical implant device of claim 1, the medical implant device formed as an intramedullary (IM) nail.

3. The medical implant device of claim 1, the at least one energy-harvesting strain sensor comprising a piezoelectric polyvinyl fluoride (PVDF) film sensor.

4. The medical implant device of claim 1, the at least one electronic element comprising at least one of a capacitor or a floating gate (FG) circuit.

5. The medical implant device of claim 4, the sensor information comprising at least one of:
capacitor information associated with the capacitor indicating a charge stored on the capacitor,
FG information associated with the FG circuit indicating at least one of a charge stored on the FG circuit, a number of channels storing a charge, or an injection rate, or
telemetry charge information indicating a charge status of the telemetry device.

6. The medical implant device of claim 1, the at least one energy-harvesting strain sensor comprising a piezoelectric polyvinyl fluoride (PVDF) film sensor electrically coupled to a floating gate (FG) circuit, the charge to power the FG circuit.

7. The medical implant device of claim 1, the medical implant device comprising an intramedullary (IM) nail having a cannulation, the at least one energy-harvesting strain sensor comprising a film arranged within the cannulation.

8. The medical implant device of claim 7, the at least one energy-harvesting strain sensor formed as a cylinder to correspond to the cannulation, the cylinder comprising a flexible polyimide circuit and electrode layers surrounding the at least one energy-harvesting strain sensor formed from a piezoelectric polyvinyl fluoride (PVDF) film.

9. The medical implant device of claim 1, comprising a cap configured to store the at least one electronic element electrically coupled to the at least one energy-harvesting strain sensor

10. The medical implant device of claim 1, comprising:
a pocket formed in an outer surface of the medical implant device, the at least one energy-harvesting strain sensor arranged within the pocket; and
a lid to hermetically seal the pocket.

11. A method of determining a healing status of a bone structure of a patient, the method comprising:
determining a step count for the patient;
determining sensor information associated with at least one energy-harvesting strain sensor of a medical implant device in contact with the bone structure, the at least one energy-harvesting strain sensor operative to:
generate a charge responsive to a strain force on the medical implant device, and
provide at least a portion of the charge to at least one electronic element, the at least one electronic element comprising a telemetry device operably coupled to the at least one energy-harvesting sensor, the telemetry device powered, at least in part, by the at least a portion of the charge to transmit the sensor information associated with the charge to a receiver device; and
determining the healing status based on the step count and the sensor information.

12. The method of claim 11, the at least one electronic element comprising at least one of a capacitor or a floating gate (FG) circuit.

13. The method of claim 13, the sensor information comprising at least one of:
capacitor information associated with the capacitor indicating a charge stored on the capacitor,
FG information associated with the FG circuit indicating at least one of a charge stored on the FG circuit, a number of channels storing a charge, or an injection rate, or
telemetry charge information indicating a charge status of the telemetry device

14. The method of claim 14, comprising:
determining at least one value of expected sensor information for the step count based on healing information; and
determining the healing status as a healing stage corresponding to the at least one expected value of sensor information.

15. The method of claim 13, the at least one value of expected sensor information comprising at least one of a capacitor charge, a telemetry device charge status, a number of charged FG channels, or an FG injection rate.

16. An apparatus, comprising:
a storage device; and
logic, at least a portion of the logic implemented in circuitry coupled to the storage device to implement a healing status analysis process, the logic to:
determine a step count for the patient;
determine sensor information associated with at least one energy-harvesting strain sensor of a medical implant device in contact with the bone structure, the at least one energy-harvesting strain sensor operative to:
generate a charge responsive to a strain force on the medical implant device, and
provide at least a portion of the charge to at least one electronic element, the at least one electronic element comprising a telemetry device operably coupled to the at least one energy-harvesting sensor, the telemetry device powered, at least in part, by the at least a portion of the charge to transmit the sensor information associated with the charge to a receiver device; and
determine the healing status based on the step count and the sensor information.

17. The apparatus of claim 16, the at least one electronic element comprising at least one of a capacitor or a floating gate (FG) circuit.

18. The apparatus of claim 17, the sensor information comprising at least one of:
capacitor information associated with the capacitor indicating a charge stored on the capacitor,
FG information associated with the FG circuit indicating at least one of a charge stored on the FG circuit, a number of channels storing a charge, or an injection rate, or
telemetry charge information indicating a charge status of the telemetry device

19. The apparatus of claim 18, the logic to:
determine at least one value of expected sensor information for the step count based on healing information, and
determine the healing status as a healing stage corresponding to the at least one expected value of sensor information.

20. The apparatus of claim 19, the at least one value of expected sensor information comprising at least one of a capacitor charge, a telemetry device charge status, a number of charged FG channels, or an FG injection rate.
